(19) United States
(12) Patent Application Publication
(10) Pub. No.: US 2025/0299801 A1
(43) Pub. Date: Sep. 25, 2025

(54) CLASSIFICATION OF CANCER FOR TREATMENT AND/OR MANAGEMENT BASED ON MACHINE LEARNING MODELS

(71) Applicant: Technion Research & Development Foundation Limited, Haifa (IL)

(72) Inventors: Ron KIMMEL, Haifa (IL); Gil SHAMAI, Haifa (IL)

(73) Assignee: Technion Research & Development Foundation Limited, Haifa (IL)

(21) Appl. No.: 19/175,046

(22) Filed: Apr. 10, 2025

Related U.S. Application Data
(63) Continuation of application No. 19/084,930, filed on Mar. 20, 2025, now abandoned.
(60) Provisional application No. 63/567,968, filed on Mar. 21, 2024.

(51) Int. Cl. G16H 30/40; G06N 20/00; G16H 10/40; G16H 20/10; G16H 50/20; G16H 50/70

(52) U.S. Cl. CPC G16H 30/40; G06N 20/00; G16H 10/40; G16H 20/10; G16H 50/20; G16H 50/70

(57) ABSTRACT

There is provided a method of classifying cancer, comprising: feeding an image of a histology slide of a cancer into a machine learning (ML) model, obtaining a score indicative of a probability of a positive status or a negative status of a marker from the ML model, accessing an indication of the positive status or the negative status of the marker obtained by a laboratory test, computing a threshold for determining whether the score generated by the ML model is discordant with respect to the indication according to the laboratory test, in response to the score being greater than a threshold and the negative status of the marker according to the laboratory test, classifying the cancer as a first category, and in response to the score being less than the threshold and the positive status of the marker according to the laboratory test, classifying the cancer as a second category.

### RELATED APPLICATIONS

This application is a Continuation of U.S. patent application Ser. No. 19/084,903 filed on Mar. 20, 2025, which claims the benefit of priority under 35 USC § 119 (e) of U.S. Provisional Patent Application No. 63/567,968 filed on Mar. 21, 2024. The contents of the above applications are all incorporated by reference as if fully set forth herein in their entirety.

### BACKGROUND

The present invention, in some embodiments thereof, relates to cancer classification and, more specifically, but not exclusively, to approaches based on machine learning models for classification of cancer, for example, for treatment and/or management.

Some cancer cells, such as breast cancer, may express receptors on their surfaces. Treatment for cancer may depend on whether the cancer expresses the receptors or not. For example, breast cancer may be classified as estrogen receptor (ER) positive or negative. Treatment may differ for breast cancer that is ER positive versus cancer that is ER negative. ER positive breast cancer may be treated using hormone therapy that blocks the estrogen receptors or lowers estrogen levels in the body, which can slow down or stop the growth of cancer cells.

### SUMMARY

According to a first aspect, a computer implemented method of classifying a cancer for treatment and/or management thereof, comprising: feeding an image of a histology slide of a cancer obtained from a subject into a machine learning model, obtaining a score indicative of a probability of a positive status or a negative status of a marker associated with a targeted treatment from the machine learning (ML) model, accessing an indication of the positive status or the negative status of the marker obtained by a laboratory test for presence of the marker on the cancer, computing a threshold for determining whether the score generated by the ML model is discordant with respect to the indication determined according to the laboratory test, in response to the score being equal to or greater than a threshold and the negative status of the marker according to the laboratory test, classifying the cancer as a first category, and in response to the score being less than the threshold and the positive status of the marker according to the laboratory test, classifying the cancer as a second category.

According to a second aspect, a system for classifying a cancer for treatment and/or management thereof, comprising: at least one processor executing a code for: feeding an image of a histology slide of a cancer obtained from a subject into a ML model, obtaining a score indicative of a probability of positive status or negative status of a marker associated with a targeted treatment from the ML model, accessing an indication of the positive status or the negative status of the marker obtained by a laboratory test for presence of the marker on the cancer, computing a threshold for determining whether the score generated by the ML model is discordant with respect to the indication determined according to the laboratory test, in response to the score being equal to or greater than a threshold and the negative status of the marker according to the laboratory test, classifying the cancer as a first category, and in response to the score being less than the threshold and positive status of the marker according to the laboratory test, classifying the cancer as a second category.

According to a third aspect, a non-transitory medium storing program instructions for classifying a cancer for treatment and/or management thereof, which when executed by at least one processor, cause the at least one processor to: feed an image of a histology slide of a cancer obtained from a subject into a ML model, obtain a score indicative of a probability of positive status or negative status of a marker associated with a targeted treatment from the ML model, access an indication of the positive status or the negative status of the marker obtained by a laboratory test for presence of the marker on the cancer, compute a threshold for determining whether the score generated by the ML model is discordant with respect to the indication determined according to the laboratory test, in response to the score being equal to or greater than a threshold and the negative status of the marker according to the laboratory test, classify the cancer as a first category, and in response to the score being less than the threshold and the positive status of the marker according to the laboratory test, classify the cancer as a second category.

In a further implementation form of the first, second, and third aspects, the marker is selected from: a molecular biomarker, a genomic marker, and a prognostic marker.

In a further implementation form of the first, second, and third aspects, the cancer comprises breast cancer, the marker comprises an estrogen receptor (ER), and targeted treatment is designed for blocking and/or interfering with function of the ER, wherein the targeted treatment is selected from: selective estrogen receptor modulator, aromatase inhibitor, ovarian suppression therapy, AKT inhibitors, CDK 4/6 inhibitors, mTor inhibitors, PI3K inhibitors, and antibody-drug conjugates (ADCs).

In a further implementation form of the first, second, and third aspects, the cancer, marker, and target treatment are selected from the following sets: {breast, HER2, HER2 targeted therapy selected from trastuzumab, pertuzumab, lapatinib, and trastuzumab deruxtecan}, {breast, the marker is determined via the laboratory test of oncotypeDx recurrence score, low ODX scores are treated with endocrine therapy alone and high ODX scores are treated with the addition of chemotherapy}, {prostate, national comprehensive cancer network (NCCN) risk classification, low-risk is managed with active surveillance, intermediate- and high-risk are treated with radical prostatectomy and/or external beam radiation therapy (EBRT) and/or or brachytherapy and/or androgen deprivation therapy (ADT), {lung, epidermal growth factor receptor (EGFR), tyrosine kinase inhibitors (TKIs)}, {colon, microsatellite instability (MSI), immune checkpoint inhibitors}, {lung or melanoma or head and neck or bladder, programmed death-ligand 1 (PD-L1), immune checkpoint inhibitors}, {non-small cell lung cancer, ALK/ROS1 rearrangements, ALK or ROS1 inhibitors}, {prostate cancer, androgen receptor (AR), androgen deprivation therapy (ADT)}, {melanoma or color, BRAF mutation, BRAF inhibitors and/or MEK inhibitors}, {solid tumor, tumor mutational burden (TMB), immune checkpoint inhibitors}, {breast cancer, mammaprint score, adjuvant chemotherapy}, {neuroendocrine tumors, Ki-67, platinum-based chemotherapy}.

In a further implementation form of the first, second, and third aspects, the first category indicates that the cancer is likely to respond to the targeted treatment despite negative status of the marker, and the second category indicates that the cancer is unlikely to respond to the targeted treatment despite positive status of the marker.

In a further implementation form of the first, second, and third aspects, further comprising in response to the classifying the cancer as the first category, treating the subject using the targeted treatment.

In a further implementation form of the first, second, and third aspects, further comprising in response to the classifying the cancer as the second category, treating the subject with a second treatment predicted to be effective for the cancer, wherein the second treatment excludes the targeted treatment.

In a further implementation form of the first, second, and third aspects, further comprising in response to the classifying the cancer as the first category, excluding the subject from a clinical trial with inclusion criteria indicating negative status of the molecular biomarker.

In a further implementation form of the first, second, and third aspects, further comprising in response to the classifying the cancer as the second category, excluding the subject from a clinical trial with inclusion criteria indicating positive status of the molecular biomarker.

In a further implementation form of the first, second, and third aspects, the image excludes visual depiction of the marker associated with the targeted treatment.

In a further implementation form of the first, second, and third aspects, the laboratory test is based on visual depiction of the marker associated with the targeted treatment.

In a further implementation form of the first, second, and third aspects, the histology slide includes a slice of the cancer stained with a hematoxylin and eosin (H&E) stain.

In a further implementation form of the first, second, and third aspects, the laboratory test includes immunohistochemistry.

In a further implementation form of the first, second, and third aspects, further comprising: creating a training dataset of a plurality of records, wherein a record includes a sample histology slide of the cancer obtained from a sample subject, and a ground truth indicating positive status or negative status of the marker obtained according to the laboratory test, and training the machine learning model on the training dataset.

In a further implementation form of the first, second, and third aspects, wherein: the marker comprises a prognostic marker, obtaining the score of the positive status or negative status comprises obtaining a predicted survival time as an outcome of the machine learning model, wherein accessing comprises accessing a prediction of the survival time based on the laboratory test, and in response to the cancer being classified as the first category or second category, providing the predicted survival time generated by the machine learning model in place of the prediction of the survival time based on the laboratory test.

In a further implementation form of the first, second, and third aspects, further comprising: creating a training dataset of a plurality of records, wherein a record includes a sample histology slide of the cancer obtained from a sample subject, and a first ground truth indicating positive status or negative status of the marker obtained according to the laboratory test, and a second ground truth indicating survival time, and training the machine learning model on the training dataset.

In a further implementation form of the first, second, and third aspects, the machine learning model is only fed the image of the histology slide of the cancer, excluding other data.

According to a fourth aspect, a computer implemented method of classifying a cancer for treatment and/or management thereof, comprises: feeding an image of a histology slide of a cancer obtained from a subject into a machine learning model, obtaining a likelihood of a positive status or a negative status of a molecular biomarker associated with a targeted treatment from the machine learning model, accessing an indication of the positive status or the negative status of the molecular biomarker obtained by a laboratory test for presence of the molecular biomarker on the cancer, in response to the likelihood being equal to or greater than a threshold and the negative status of the molecular biomarker according to the laboratory test, classifying the cancer as a first category, and in response to the likelihood being less than the threshold and the positive status of the molecular biomarker according to the laboratory test, classifying the cancer as a second category.

According to a fifth aspect, a system for classifying a cancer for treatment and/or management thereof, comprises: at least one processor executing a code for: feeding an image of a histology slide of a cancer obtained from a subject into a machine learning model, obtaining a likelihood of positive status or negative status of a molecular biomarker associated with a targeted treatment from the machine learning model, accessing an indication of positive status or negative status of the molecular biomarker obtained by a laboratory test for presence of the molecular biomarker on the cancer, in response to the likelihood being equal to or greater than a threshold and the negative status of the molecular biomarker according to the laboratory test, classifying the cancer as a first category, and in response to the likelihood being less than the threshold and positive status of the molecular biomarker according to the laboratory test, classifying the cancer as a second category.

According to a sixth aspect, a non-transitory medium storing program instructions for classifying a cancer for treatment and/or management thereof, which when executed by at least one processor, cause the at least one processor to: feed an image of a histology slide of a cancer obtained from a subject into a machine learning model, obtain a likelihood of positive status or negative status of a molecular biomarker associated with a targeted treatment from the machine learning model, access an indication of positive status or negative status of the molecular biomarker obtained by a laboratory test for presence of the molecular biomarker on the cancer, in response to the likelihood being equal to or greater than a threshold and the negative status of the molecular biomarker according to the laboratory test, classify the cancer as a first category, and in response to the likelihood being less than the threshold and the positive status of the molecular biomarker according to the laboratory test, classify the cancer as a second category.

In a further implementation form of the fourth, fifth, and sixth aspects, the first category indicates that the cancer is likely to respond to the targeted treatment despite negative status of the molecular biomarker.

In a further implementation form of the fourth, fifth, and sixth aspects, the second category indicates that the cancer is unlikely to respond to the targeted treatment despite positive status of the molecular biomarker.

In a further implementation form of the fourth, fifth, and sixth aspects, further comprising in response to the classifying the cancer as the first category, treating the subject using the targeted treatment.

In a further implementation form of the fourth, fifth, and sixth aspects, further comprising in response to the classifying the cancer as the second category, treating the subject with a second treatment predicted to be effective for the cancer, wherein the second treatment excludes the targeted treatment.

In a further implementation form of the fourth, fifth, and sixth aspects, further comprising in response to the classifying the cancer as the first category, excluding the subject from a clinical trial with inclusion criteria indicating negative status of the molecular biomarker.

In a further implementation form of the fourth, fifth, and sixth aspects, further comprising in response to the classifying the cancer as the second category, excluding the subject from a clinical trial with inclusion criteria indicating positive status of the molecular biomarker.

In a further implementation form of the fourth, fifth, and sixth aspects, the cancer comprises breast cancer, the molecular biomarker comprises an estrogen receptor (ER), and targeted treatment is designed for blocking and/or interfering with function of the ER.

In a further implementation form of the fourth, fifth, and sixth aspects, the targeted treatment is selected from: AKT inhibitors, CDK 4/6 inhibitors, mTor inhibitors, PI3K inhibitors, and antibody-drug conjugates (ADCs).

In a further implementation form of the fourth, fifth, and sixth aspects, the image excludes visual depiction of the molecular biomarker associated with the targeted treatment.

In a further implementation form of the fourth, fifth, and sixth aspects, the laboratory test is based on visual depiction of the molecular biomarker associated with the targeted treatment.

In a further implementation form of the fourth, fifth, and sixth aspects, the histology slide includes a slice of the cancer stained with a hematoxylin and eosin (H&E) stain.

In a further implementation form of the fourth, fifth, and sixth aspects, the laboratory test includes immunohistochemistry.

In a further implementation form of the fourth, fifth, and sixth aspects, the likelihood is a continuous variable within a range.

In a further implementation form of the fourth, fifth, and sixth aspects, further comprising: creating a training dataset of a plurality of records, wherein a record includes a sample histology slide of the cancer obtained from a sample subject, and a ground truth indicating positive status or negative status of the molecular biomarker obtained according to the laboratory test, and training the machine learning model on the training dataset.

In a further implementation form of the fourth, fifth, and sixth aspects, further comprising: obtaining a predicted survival time as an outcome of the machine learning model.

In a further implementation form of the fourth, fifth, and sixth aspects, further comprising: creating a training dataset of a plurality of records, wherein a record includes a sample histology slide of the cancer obtained from a sample subject, and a first ground truth indicating positive status or negative status of the molecular biomarker obtained according to the laboratory test, and a second ground truth indicating survival time, and training the machine learning model on the training dataset.

In a further implementation form of the fourth, fifth, and sixth aspects, further comprising: in response to the likelihood being equal to or greater than a threshold and the positive status of the molecular biomarker according to the laboratory test, classifying the cancer as a third category, and in response to the likelihood being less than the threshold and the negative status of the molecular biomarker according to the laboratory test, classifying the cancer as a fourth category.

In a further implementation form of the fourth, fifth, and sixth aspects, a higher value of the likelihood denotes a better predicted outcome for the subject, and a lower values of the likelihood denotes a worse predicted outcome for the subject.

In a further implementation form of the fourth, fifth, and sixth aspects, the machine learning model is only fed the image of the histology slide of the cancer, excluding other data.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 a block diagram of components of a system 100 for classifying a cancer depicted in a histological image, in accordance with some embodiments of the present invention;

FIG. 2 is a flowchart of a method of classifying a cancer depicted in a histological image, in accordance with some embodiments of the present invention;

FIG. 3 is a flowchart of a method of training a machine learning model for processing histological images, in accordance with some embodiments of the present invention;

FIG. 4, which includes graphs comparing the gene expression of four groups: ER-negative patients, ER-positive patients, ER-negative AI-high, and ER-positive AI-low, based on an experiment conducted by Inventors, in accordance with some embodiments of the present invention;

FIGS. 5A-5D are graphs comparing patient prognosis for ER status and predicted morphological signal, based on an experiment conducted by Inventors, in accordance with some embodiments of the present invention;

FIG. 6 is a graph indicating cases that were discordant between AI predictions and IHC results in an experiment conducted by Inventors, in accordance with some embodiments of the present invention; and

FIGS. 7A-7F are graphs comparing gene expression-based scores within each quantile between IHC-determined ER-positive and ER-negative cases as part of an experiment conducted by Inventors, in accordance with some embodiments of the present invention.

### DETAILED DESCRIPTION

The present invention, in some embodiments thereof, relates to cancer classification and, more specifically, but not exclusively, to a machine learning models for classification of cancer for treatment and/or management and/or prediction of prognosis (e.g., survival time).

As used herein, the terms machine learning (ML) model and artificial intelligence (AI) model are used interchangeably.

As used herein, the terms histological image and image of a histology slide are used interchangeably.

As used herein, the terms likelihood and probability may sometimes be interchanged.

As used herein, the term marker may refer, for example, to a biomarker, molecular biomarker, genetic marker, genomic marker, prognostic marker, predictive molecular marker, and the like. The aforementioned terms may be interchanged accordingly, and are not meant to be necessarily limiting. For example, the molecular biomarker may be interchanged with the term genomic biomarker, and/or may be interchanged with the term marker—i.e., features relates to the molecular biomarker are not meant to be necessarily limited to molecular biomarkers.

An aspect of some embodiments of the present invention relates to systems, methods, computing devices, and/or code instructions (stored on a data storage device and executable by one or more processors) for identifying subjects with atypical marker profiles, where expression of the marker deviates from expected clinical patterns. In particular, where predictions by a machine learning model analyzing a histological image of tissue of the subject indicating a state of the marker are discordant with results of a laboratory test designed to detect the marker.

Discordant cases may refer to different results obtained according to a machine learning model generating a score indicative of a marker based on an input of a histological image of tissue of a subject, and a laboratory analysis for the presence of the marker in the tissue. A threshold may be computed. The threshold may be used for classifying the cancer according to the score generated by the ML model. The threshold may be used for determining whether the score generated by the ML model is discordant with respect to the outcome determined according to the laboratory test, for classifying the cancer into a first category or second category, as described herein. For examples, scores generated by the ML model above the threshold are classified as “positive” and scores below the threshold as “negative.” The “positive” or “negative” outcomes determined by applying the threshold to the scores generated by the ML model may be compared to the “positive” or “negative” outcomes obtained based on the laboratory test, to determine whether the cancer is a discordant case, and classified into the first category or second category, as described herein. Classification of the cancer (or subject) as discordant may refer to classification into the first category or second category. As described herein, Inventors discovered that the first category and second category represent unique cases of marker expression by the cancer, which may be managed in a way that is contrary to standard clinical practice.

An aspect of some embodiments of the present invention relates to systems, methods, computing devices, and/or code instructions (stored on a data storage device and executable by one or more processors) for classifying a cancer for treatment and/or management thereof. An image of a histology slide of a cancer obtained from a subject is fed into a machine learning model. The histology slide may include a slice of the cancer, for example, obtained via a biopsy and/or surgical resection. The image excludes visual depiction of a molecular biomarker associated with the targeted treatment and/or prognostic marker. The cancer depicted in the image may be stained with a stain that is not designed to visually indicate molecular biomarker status, for example, a hematoxylin and eosin (H&E) stain. A likelihood (e.g., probability) of a positive status of a molecular biomarker being associated with a targeted treatment is obtained from the machine learning model. The likelihood may be represented as a score (also referred to herein as AI score), optionally indicating a probability, optionally a continuous value within a range, for example, from 0 to 1, or 0 to 100, or other ranges. An indication of positive status of the molecular biomarker obtained by a laboratory test for presence of the molecular biomarker on the cancer, is accessed. The laboratory test may be based on visual depiction of the molecular biomarker associated with the targeted treatment, for example, as described herein. In response to the likelihood being equal to or greater than a threshold and negative status of the molecular biomarker according to the laboratory test, the cancer is classified as a first category. In response to the likelihood being less than the threshold and positive status of the molecular biomarker according to the laboratory test, the cancer is classified as a second category.

The first and second categories are different than standard classification of cancer in which cancer is classified as either molecular biomarker positive or molecular biomarker negative, for example, where molecular biomarker positive cancer is likely to respond to the targeted treatment and molecular biomarker negative cancer is unlikely to respond to the targeted treatment. The first category may indicate that the cancer is likely to respond to the targeted treatment, despite the negative status of the molecular biomarker. The second category may indicate that the cancer is unlikely to respond to the targeted treatment, despite the positive status of the molecular biomarker. Additional description supporting the aforementioned features related to the first category and the second category is provided, for example, with respect to the experiment described in the Examples section below.

Optionally, in response to the cancer being classified into the first category, the subject may be treated using the targeted treatment. Alternatively, in response to the cancer being classified into the second category, the subject may be treated with a second treatment predicted to be effective for the cancer, where the second treatment excludes the targeted treatment. It is noted that these approach may be in contrast to standard practice where treatment is based on the molecular biomarker positive or negative status of the cancer.

Alternatively or additionally, in response to the cancer being classified into the first category, the subject may be excluded from a clinical trial with inclusion criteria indicating negative status of the molecular biomarker, for example, for testing a new therapy for patients with molecular biomarker negative cancer. Alternatively, in response to the cancer being classified into the second category, the subject may be excluded from a clinical trial with inclusion criteria indicating positive status of the molecular biomarker, for example, for testing a new therapy for patients with molecular biomarker positive cancer. It is noted that these approach may be in contrast to standard practice where inclusion or exclusion in clinical trials is based on the molecular biomarker positive or negative status of the cancer.

Alternatively or additionally, in response to the cancer being classified into the first category or the second category, where the marker is implemented as a prognostic marker, the survival predicted by the machine learning model may be provided over the survival predicted based on the laboratory test.

At least some embodiments described herein address the technical problem and/or medical problem of tools for improving classification of cancer, for example, for improved treatment and/or management. The technical problem being solved relates to existing cancer biomarker systems failing to capture functional activity of pathways related to the biomarkers, leading to inappropriate treatment decisions in some subjects. Inventors discovered that in a subset of subjects, the machine learning model analyzing histological images of tissue depicts a “real-time” or “actual” state of the molecular pathways associated with the marker, such as whether the molecular pathways are active or not or amount of activity. In contrast, the laboratory test provides a visual indication of whether the marker is present or not without indicating the state of the molecular pathways. Subjects that are being treated using the results of the laboratory test may be inappropriately treated in cases where predictions by the machine learning model analyzing a histological image of tissue of the subject indicating a state of the marker are discordant with the results of the laboratory test designed to detect the marker. At least one embodiment described herein solves this problem by using the machine learning model to identify discordant cases which reveal previously unrecognized tumor subtypes (referred to herein as a first and second classification category). As described herein, in such discordant cases, Inventors recommend treating the subject by treating discordance between the results of the machine learning model and the laboratory test as new cancer type categories with their own treatment planning (which may not respond the same as using only the laboratory results) rather than the traditional approach of treating the subject according to the results of the laboratory test and ignoring the results of the machine learning model in the discordant cases. The discordance cases should be referred to as representing a tumor new subtype which may not have the same response to treatment as the other non-discordance cases. Moreover, as described herein, using the machine learning model results in contrast to the traditional approach of using the laboratory cases in the identified discordant cases may impact other clinical outcomes, for example, impacting inclusion/exclusion criteria of clinical trials, prediction of prognosis, and the like.

At least some embodiments described herein improve the technical field and/or medical field of tools for improving classification of cancer, for example, for improved treatment and/or management.

At least some embodiments described herein improve upon existing approaches for classification of cancer, which are based on positive or negative status of molecular biomarkers, for example, expression or lack of expression of specific receptors, by the cancer cells.

Integrating molecular biomarkers into the clinical decision making process has significantly advanced cancer diagnosis and treatment, helping clinicians select therapies and stratify patients by prognosis. Examples of such biomarkers include estrogen receptor (ER)9 and the OncotypeDX score in breast cancer10. These markers and signatures, typically assessed via IHC, RNA sequencing, and other molecular tests, aid in selecting targeted therapies and assessing prognosis11.

However, standard assessments by traditional molecular methods (i.e., current molecular assessments) like immunohistochemistry (IHC) often fall short of capturing the full complexity of cancer biology, resulting in cases where patients deemed eligible for targeted therapies do not respond, while other patients, classified as ineligible, might benefit¹², and/or in cases classified has having high or low prognosis and experiencing the opposite. This can result in missed therapeutic opportunities or ineffective treatments that add costs, toxicity, and unnecessary burden. Emerging evidence suggests that cancer involves complex layers beyond the measured expression of biomarkers. Tumors may express a marker without activating the relevant pathway, leading to insensitivity to targeted therapies, or show pathway activity despite low marker expression. These complexities suggest that standard biomarker assessments may be insufficient and that more comprehensive tumor characterization approaches are needed¹³.

Hematoxylin and eosin (H&E) staining, a fundamental method in pathology, is widely accessible and routinely applied to solid tumors for assessing tissue morphology. Deep learning models applied to digitized H&E-stained slides may predict presence of molecular biomarkers, such as protein expression and genetic mutations^{3,4,7,8,14}. These models appear to offer a rapid, cost-effective approach to biomarker assessment without using traditional testing methods. However, despite improvements over the years, as these H&E-derived predictions are inferior approximations to traditional molecular tests, their clinical utility has been very limited. Therefore, these H&E based predictions by models have not been adopted into clinical practice and cannot replace convention laboratory biomarker testing. Thus, they are largely viewed as low-cost approximations to traditional molecular tests, limiting their clinical integration. These sub-optimal performances represent discrepancies between the models' predictions and actual molecular test results. As described herein, for example, in the Examples section below, Inventors discovered that discrepancies between conventional molecular biomarker laboratory test results and their deep learning-based (e.g., AI based) predictions from H&E images do not necessarily reflect errors of the models. Rather, AI-derived predictions from H&E images appear to capture additional biologically and/or clinically relevant information beyond marker presence alone. These discrepancies appear to reflect meaningful information on downstream pathway activation and/or the tumor's functional state, potentially offering new predictive insights. For instance, in certain ER-positive breast cancer cases with inactive ER pathways, tumor morphology may align more closely with ER-negative features, leading AI to predict ER-negative status H&E slides. Such cases often exhibit a lack of response to hormone therapy, suggesting that even though the AI's prediction was wrong with respect to its ER-status prediction task, the AI may have captured a relevant signal with predictive information additional to the molecular-based ER-status. At least one embodiment described herein relates to utilizing these discrepancies for refining clinical decisions. These embodiments may potentially transform the role of digital pathology in clinical oncology and/or contribute to more precise, personalized cancer care.

Although significant progress has being made, with whole-slide images (WSIs) of H&E-stained tissue being utilized to diagnose tumors, predict receptor status4,7,8, identify genetic mutations1⁴, patient survival1⁵, and genomic-based signatures6, and some studies have also predicted therapy response using H&E images combined with clinical variables1⁷, results have not been sufficiently accurate to be utilized in clinical practice. Directly predicting therapy response or disease progression from H&E data requires extensive matched imaging and follow-up datasets, specific to each drug or treatment. Such data remains limited, impacting the applicability and generalizability of these models across cohorts. Moreover, without using data from randomized clinical trials, predicting patient prognosis would not provide predictive information that could change clinical decisions. In contrast, at least one embodiment described herein provides the potential advantage of bypassing the need for large-scale response and/or gene expression data by using traditional markers to derive new morphological information.

At least some embodiments described herein provide solutions for the aforementioned technical problem, and/or improve the aforementioned technical field and/or medical field, and/or improve upon existing approaches, by classifying cancer into a first category or second category according to likelihood of positive status or negative status of a molecular biomarker obtained from a machine learning model fed an image of a histology slide of the cancer and an indication of positive status or negative status of the molecular biomarker obtained by a laboratory test. The cancer is classified into the first category in response to the likelihood being equal to or greater than a threshold and negative status of the molecular biomarker according to the laboratory test. The cancer is classified into the second category in response to the likelihood being less than the threshold and positive status of the molecular biomarker determined according to the laboratory test.

Inventors trained deep learning models to predict the ER status (absence or presence of molecular biomarkers) in breast cancer from H&E images, achieving a performance AUC of 0.91-0.94 per patient. Inventors discovered that the morphological patterns detected by the models for predicting ER status enable the stratification of cancer into subtypes different than those stratified by the ER status. Namely, ER-positive patients who were ‘falsely’ predicted as ER-negative by the model (i.e., displayed morphological patterns similar to ER-negative patients), had a different genetic expression and prognosis than the rest of the ER-positive patients, and vice versa. Inventors hypothesize that the models predict the tumor's ER dependency of the tumor rather than its ER status. Extending the models to other molecular biomarkers led to similar conclusions. These discoveries made by Inventors suggest that deep learning models reach a performance plateau in predicting molecular biomarker positive or negative status because they converge to identifying other distinct, meaningful tumor subtypes. Furthermore, Inventors' discoveries suggest a new use of deep learning for tumor subtyping and patient stratification to targeted therapy.

Inventors' findings, for example as described in the Examples section below, may indicate that while the deep-learning models were trained to predict ER status, they may have actually identified a distinct tumor subtype. This new subtype may differ from the conventional ER status in terms of genetic expression and/or prognosis. The survival analysis described in the Examples section may suggest that the AI models predict tumor ER dependency rather than mere ER status. More particularly, some tumors may exhibit ER positivity, but due to for example complex genetic pathways inhibiting receptor activation, they may behave like ER-negative tumors. Their morphology may therefore resemble that of ER-negative tumors. Conversely, some ER-negative tumors may function like ER-positive tumors, with morphologies reflective of ER positivity. The models may predict ER dependency by focusing on tumor morphology, even though they were initially trained to predict ER status as measured by IHC. This observation by Inventors may also explain the performance plateau observed in recent studies regarding ER status prediction from H&E images.

These discoveries made by Inventors (e.g., as detailed in the Examples section below) may carry significant clinical implications. The ER status of a tumor is a critical determinant of hormonal therapy effectiveness. Patients who are ER-positive but AI-low might be ER-independent and unresponsive to hormone therapy. Similarly, ER-negative but AI-high patients could have tumors behaving like ER-positive tumors, potentially responding to hormone therapy. At least some embodiments described herein may be used to provide crucial insights into patient responsiveness to therapy. Preliminary results of experiments conducted by Inventors, replicated for other molecular biomarkers, showed similar trends, where AI scores not matching the trained biomarker status correlated with distinct genetic expressions and prognoses.

Other examples of use cases for classification of cancer of subjects into the first or second categories (or into the third and/or fourth categories) include:
Reporting the class of these patients in the pathology report for the oncologist.
Closely following these patients.
Considering re-diagnosis and/or considering diagnosing their biomarker status in other ways. For example, if a patient is ER-negative and likelihood-high, measuring the patient's ESR1 gene expression may be considered to see if it is expressive.
Exclusion from clinical trials.
Inclusion in clinical trials.
Repeating the laboratory test.
Using a different gene-expression based test.

For receptors like HER2, the predicted AI score might predict something different than the HER2 dependency. Thus, patients classified into the first and second categories in this case may be managed as distinct cancer subtypes and not necessarily as a false HER2 diagnoses. For example, HER2-positive AI score-low patients may still be actual HER2-positive patients with HER2-dependent cancer, but these patients may have a distinct prognosis and gene expression than the HER2-positive AI score-high patients. Overall, cancers classified into the first and second categories may be managed as distinctive cancer subtypes and/or as potentially behaving like what would be expected from the opposite biomarker status diagnosis.

Results of experiments conducted by Inventors, as described in the Examples section below, further provide evidence indicating that biomarker-positive patients with low AI scores, which can be 1-15% of the biomarker-positive patients, are less likely to benefit from the biomarker's targeted therapy compared to those with high AI scores. Hence, the AI score may be used to refine therapy eligibility, particularly when transitioning from phase 2 to phase 3 in clinical trials, potentially enhancing drug benefit endpoints.

Patients with biomarker-negative but high AI scores might benefit from therapy despite not being eligible based on biomarker status alone. Consideration of therapy for these patients could be warranted.

Patients who are biomarker-positive AI-low or biomarker-negative AI-high may exhibit significantly different prognoses and genetic expressions compared to their respective biomarker-positive or negative groups. Therefore, the AI score could serve as a prognostic and predictive biomarker, offering added value beyond the biomarker status itself.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an

Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

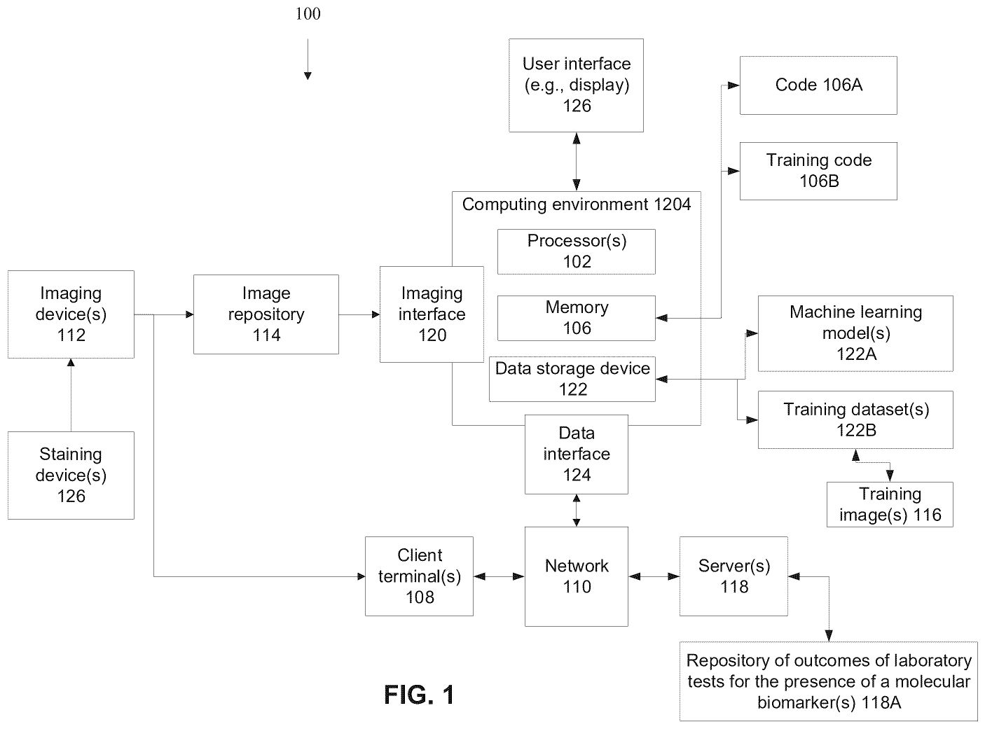

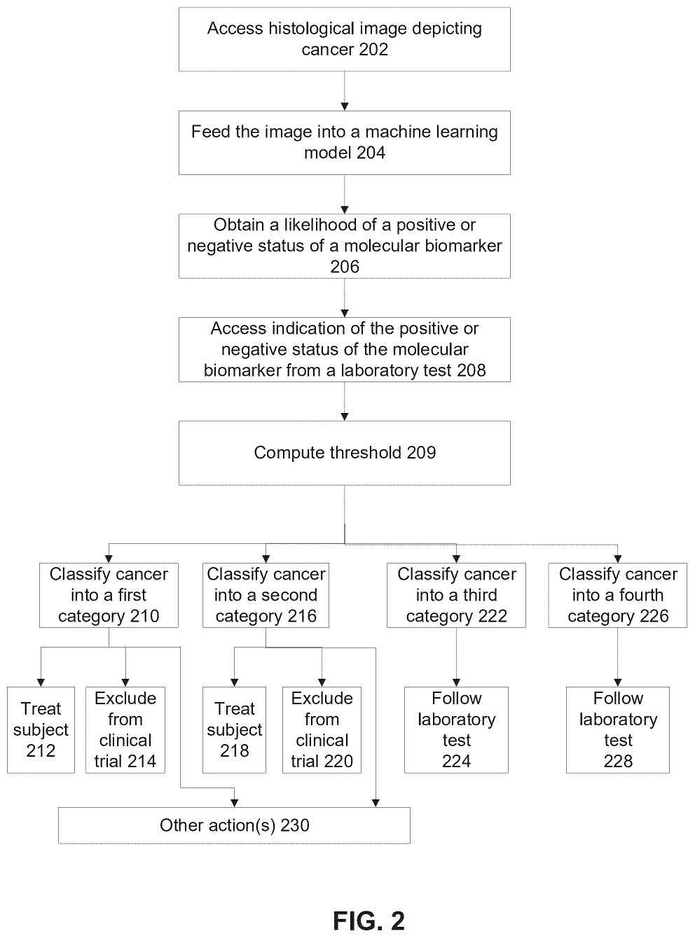

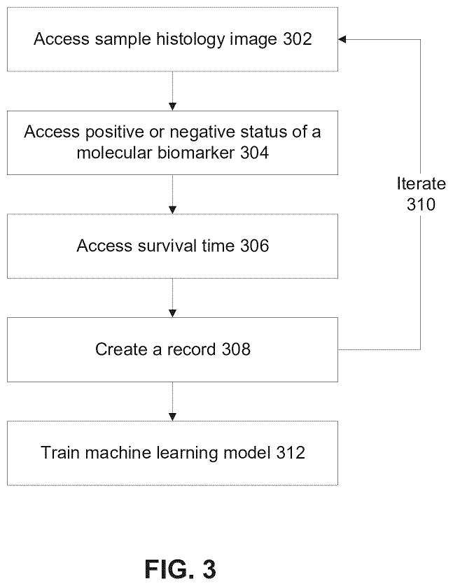

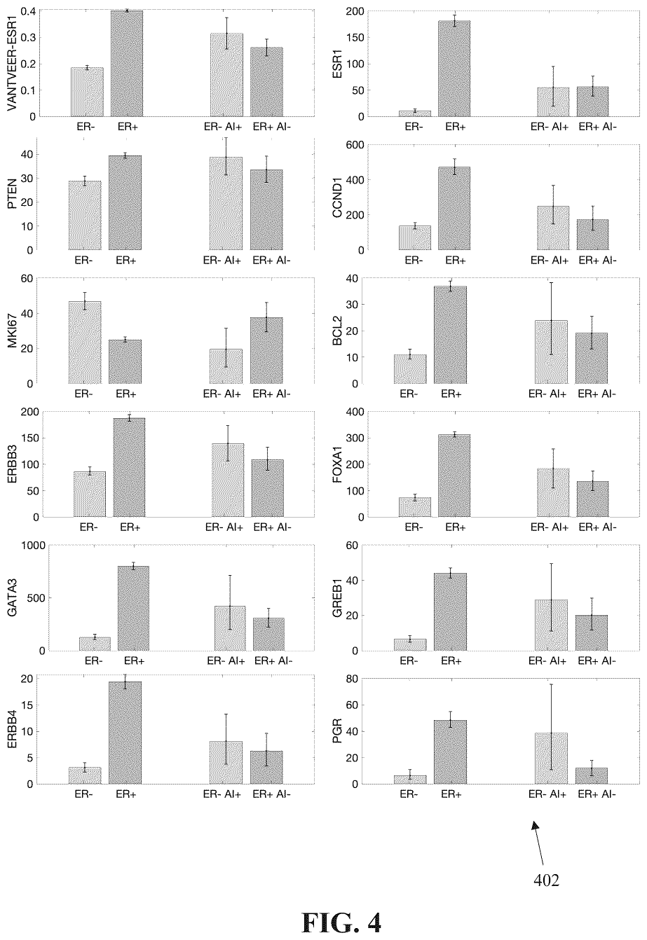

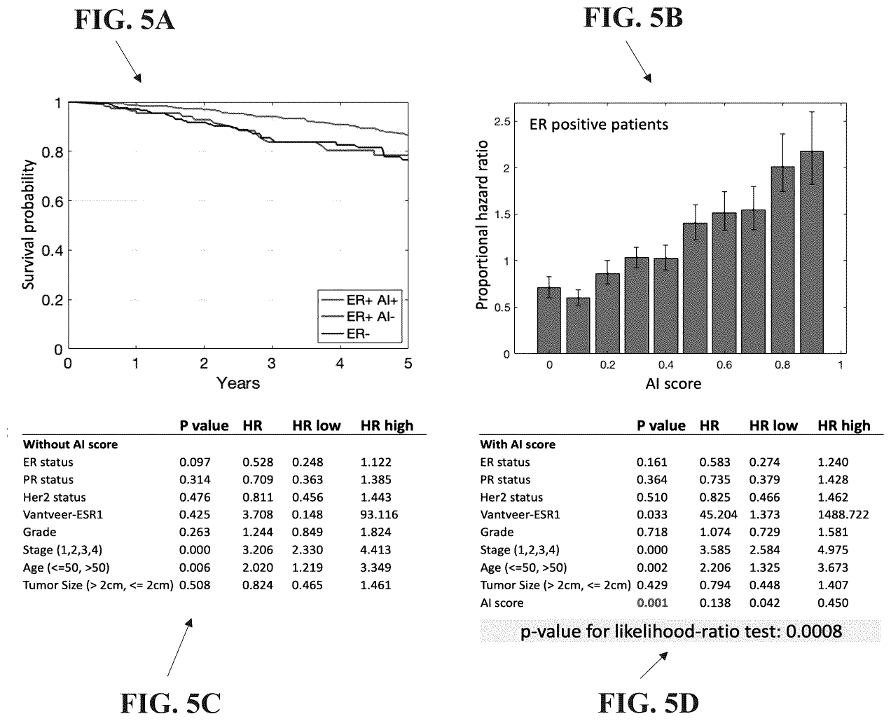

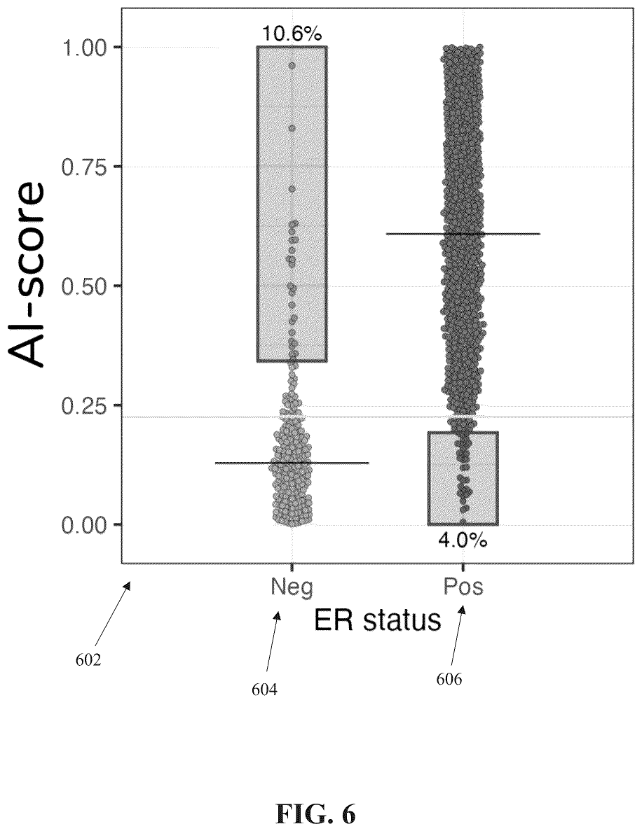

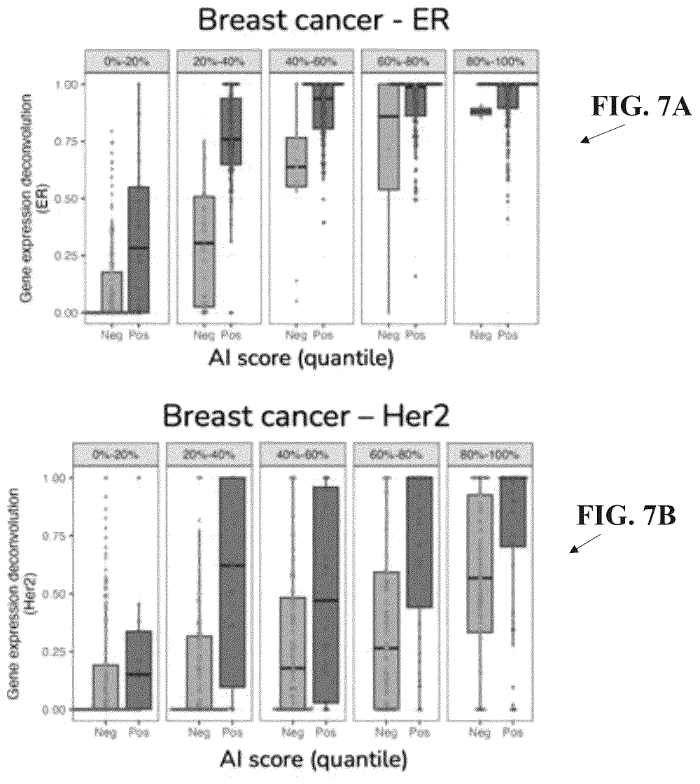

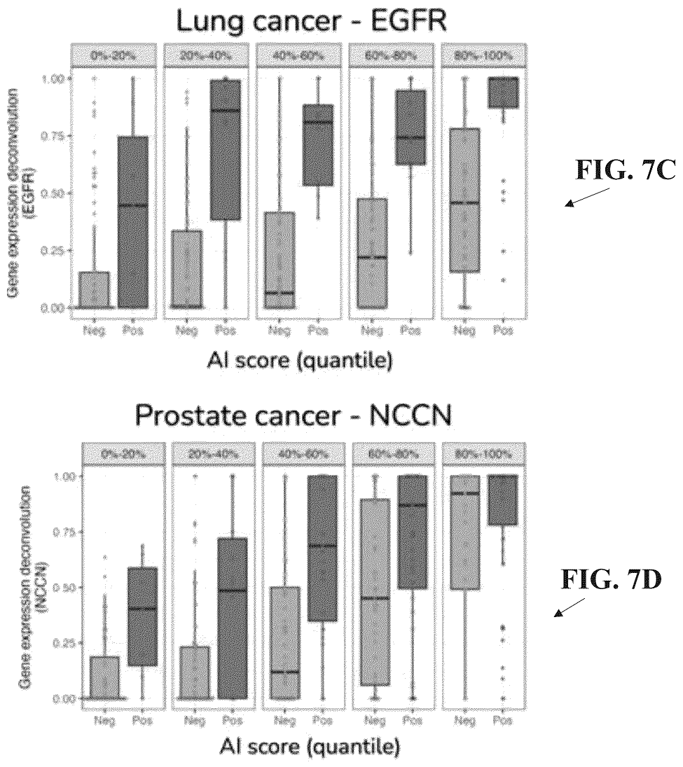

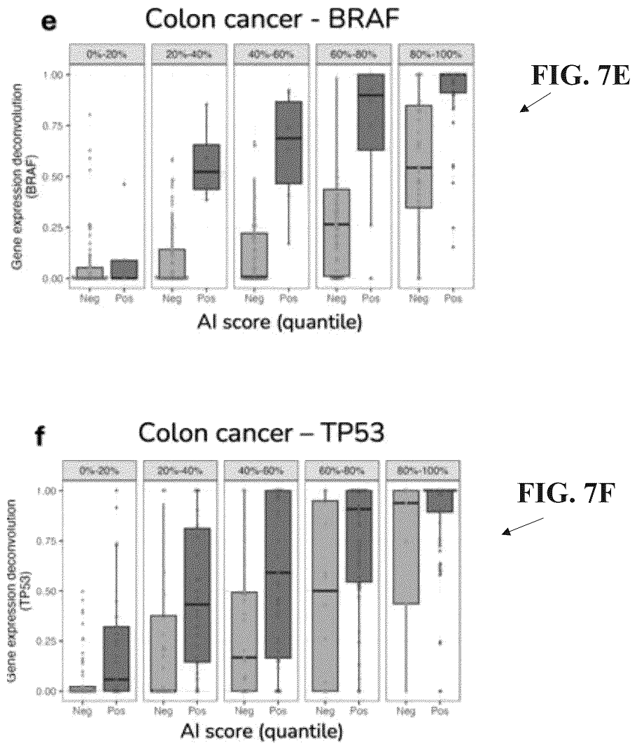

Reference is now made to FIG. 1, which is a block diagram of components of a system 100 for classifying a cancer depicted in a histological image, in accordance with some embodiments of the present invention. Reference is also made to FIG. 2, which is a flowchart of a method of classifying a cancer depicted in a histological image, in accordance with some embodiments of the present invention. Reference is also made to FIG. 3, which is a flowchart of a method of training a machine learning model for processing histological images, in accordance with some embodiments of the present invention. Referring is also made to FIG. 4, which includes graphs 402 comparing the gene expression of four groups: ER-negative patients, ER-positive patients, ER-negative AI-high, and ER-positive AI-low, based on an experiment conducted by Inventors, in accordance with some embodiments of the present invention. Referring is also made to FIGS. 5A-D, which are graphs comparing patient prognosis for ER status and predicted morphological signal, based on an experiment conducted by Inventors, in accordance with some embodiments of the present invention. Reference is also made to FIG. 6, which is a graph 602 indicating cases that were discordant between AI predictions and IHC results in an experiment conducted by Inventors, in accordance with some embodiments of the present invention. Reference is also made to FIGS. 7A-7F, which are graphs comparing gene expression-based scores within each quantile between IHC-determined ER-positive and ER-negative cases as part of an experiment conducted by Inventors, in accordance with some embodiments of the present invention.

System 100 may implement the acts of the method described with reference to the FIGs. described herein, optionally by a (hardware) processor(s) 102 of a computing environment 104 executing code instructions 106A and/or 106B stored in a memory 106.

Computing environment 104 may classify an image of a histology slide of a cancer for determining treatment and/or management. The image may be created by staining by a staining device 126 and/or captured by an imaging device 112. The image may be fed into a machine learning model(s) 122A, which may be trained on a training dataset(s) 122B, for obtaining a likelihood of a positive status or a negative status of a molecular biomarker associated with a targeted treatment. Computing environment 104 may obtain an indication of the positive status or the negative status of the molecular biomarker obtained by a laboratory test for presence of the molecular biomarker on the cancer from a repository 118A (e.g., electronic medical record), which may be hosted by a server(s) 118. Computing environment 104 classifies the cancer into a category, as described herein.

Computing environment 104 may be implemented as, for example, a client terminal, a server, a virtual server, a laboratory workstation (e.g., pathology workstation), a procedure (e.g., operating) room computer and/or server, a virtual machine, a computing cloud, a mobile device, a desktop computer, a thin client, a Smartphone, a Tablet computer, a laptop computer, a wearable computer, glasses computer, and a watch computer.

Computing 104 may include an advanced visualization workstation that sometimes is implemented as an add-on to a laboratory workstation and/or other devices for analyzing histological images, for example, for analyzing a biopsy including cancer, for guiding treatment and/or management and/or other computer added detections, to the user (e.g., pathologist, oncologist).

In an exemplary centralized implementation, computing environment 104 may be implemented as one or more servers (e.g., network server, web server, a computing cloud, virtual server) that provides centralized services (e.g., one or more of the acts described with reference to FIGs. described herein) to one or more client terminals 108 (e.g., remotely located laboratory workstations, remotely located drug discovery terminals, remote picture archiving and communication system (PACS) server, remote electronic medical record (EMR) server, remote tissue image storage server, remotely located pathology workstations, client terminal of a user such as a desktop computer) over a network 110, for example, providing software as a service (SaaS) to the client terminal(s) 108, providing an application for local download to the client terminal(s) 108, as an add-on to a web browser and/or a tissue imaging viewer application, and/or providing functions using a remote access session to the client terminals 108, such as through a web browser. In one implementation, multiple client terminals 108 each obtain respective images of histology slides of tissue with cancer from different subjects which may be captured different imaging devices 112 and/or which may be stained by the same stain or using different staining devices 126. Each of the multiple client terminals 108 provides their respective histological images to computing environment 104, and receives back a respective indication of a classification of the cancer, which may be used to guide treatment and/or management of the cancer.

Is it noted that the training of the ML model(s) 122A, and the analysis of histological images by the trained ML model(s) 122A, may be implemented by the same computing environment 104, and/or by different computing environments, for example, one computing environment trains the ML model(s) 122A, and transmits the trained ML model(s) 122A to a server for use for inference.

Computing environment 104 receives histological images captured by one or more imaging device(s) 112. Exemplary imaging device(s) 112 include: a scanner scanning in standard color channels (e.g., red, green blue), a confocal microscope with camera, and the like.

Imaging device(s) 112 may create histological images from physical tissue samples which may be obtained by a tissue extracting device, for example, a fine needle for performing fine needle aspiration (FNA), a larger bore needle for performing a core biopsy, and a cutting tool (e.g., knife, scissors, scoop) for cutting out a sample of the tissue (e.g., tumor removal).

Histological images captured by imaging machine 112 may be stored in an image repository 114, for example, a storage server, a computing cloud, virtual memory, and a hard disk. Training images 116 may be created based on the captured tissue images, by labelling the images with ground truth labels (e.g., obtained from health records of the subject, such as from server(s) 118), for example, as described herein.

Training dataset 122B may be created from training images 116 and optionally other data, as described herein. Training dataset 122B is used to train ML model(s) 122A, as described herein.

It is noted that training images 116 may be stored by a server 118, accessibly by computing environment 104 over network 110, for example, a publicly available training dataset, tissue images stored in a PACS server and/or pathology imaging server, and/or a customized training dataset created for training the ML models.

Computing environment 104 may receive the training images 116 and/or tissue images for analysis from imaging device 112 and/or image repository 114 using one or more imaging interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)). Alternatively or additionally, computing environment 104 may receive the training images 116 and/or tissue images for analysis from client terminal(s) 108 and/or server(s) 118.

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) stores code instruction for execution by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more acts and/or features of the method described with reference to the FIGs. described herein, and/or training code 106B that executes one or more acts of the method of training described herein.

Computing environment 104 may include a data storage device 122 for storing data, for example, ML model(s) 122A, and/or training dataset 122B, and/or other code and/or other data and/or other processes described herein. Data storage device 122 may be implemented as, for example, a memory, a local hard-drive, a removable storage device, an optical disk, a storage device, and/or as a remote server and/or computing cloud (e.g., accessed over network 110). It is noted that code portions of the data stored in data storage device 122 may be loaded into memory 106 for execution by processor(s) 102.

Computing environment 104 may include data interface 124, optionally a network interface, for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing environment 104 may access one or more remote servers 118 using network 110, for example, to download updated training images 116 and/or to download an updated version of the ML model(s) 122A, training code 106B, and/or the training dataset 122B.

It is noted that imaging interface 120 and data interface 124 may exist as two independent interfaces (e.g., two network ports), as two virtual interfaces on a common physical interface (e.g., virtual networks on a common network port), and/or integrated into a single interface (e.g., network interface).

Computing environment 104 may communicate using network 110 (or another communication channel, such as through a direct link (e.g., cable, wireless) and/or indirect link (e.g., via an intermediary computing environment such as a server, and/or via a storage device) with one or more of:
Client terminal(s) 108, for example, when computing environment 104 acts as a server providing services (e.g., SaaS) to remote laboratory terminals, for classifying cancer depicted in histological images.
Server 118, for example, implemented in association with a PACS and/or electronic medical record, which may host a repository of outcomes of laboratory tests for the presence of one or more molecular biomarkers 118A, as described herein.
Image repository 114 that may store training images 116 and/or histological images outputted by imaging device 112.

Computing environment 104 includes or is in communication with a user interface 126 that includes a mechanism designed for a user to enter data (e.g., personal data of the subject) and/or view the outcome of the classification of the cancer depicted in the histological image. Exemplary user interfaces 126 include, for example, one or more of, a touchscreen, a display, a keyboard, a mouse, and voice activated software using speakers and microphone.

Referring now back to FIG. 2, at 202, an image of a histology slide of a cancer (e.g., tumor) obtained from a subject is provided, for example, accessed, obtained, and/or received.

The histology image may be captured by a camera at a predefined magnification level, such as under a microscope, for example, about 20×-1000×, for example, about 200×, 400×, 600×, or 800×, or other values.

The histology image may be a whole slide image (WSI).

The histology image may be of a thin section of tissue extracted from the subject, for example, biopsy, surgical resection, and the like. The tissue may be prepared, for example, using a frozen section approach, and the like.

The histology image includes a slice of the cancer stained with a stain, for example, a hematoxylin and eosin (H&E) stain.

The histology image may exclude visual depiction of a molecular biomarker associated with a targeted treatment. The molecular biomarker may be present, but may be too small and/or may be not visible under the current illumination conditions and/or not visibly captured by the camera. For example, the histology slide is stained with H&E stain which does not depict antibody markers linked to a fluorescent dye that are visible using immunofluorescence. Alternatively, the molecular biomarker is not present in the tissue used for the histology image, but rather is used to test another sample of tissue.

The histology image may be a 2D image.

Optionally, the cancer is breast cancer, the molecular biomarker is an estrogen receptor (ER). ER may be used for determining eligibility for endocrine therapies; for hormone receptor-positive breast cancer management. The targeted treatment is designed for blocking and/or interfering with function of the ER in the body and/or for lowering estrogen levels in the body. Examples of targeted treatment for ER positive breast cancer include: selective estrogen receptor modulators (e.g., tamoxifen) and/or aromatase inhibitors (e.g., letrozole, anastrozole) in postmenopausal women, ovarian suppression may be used in premenopausal patients, AKT inhibitors, CDK 4/6 inhibitors, mTor inhibitors, PI3K inhibitors, and antibody-drug conjugates (ADCs).

Other examples of cancer(s), marker(s), and targeted treatment(s) according to the marker(s) include:
Multiple cancers (e.g., lung, melanoma, head and neck, bladder): ML models may be used for predicting PD-L1 (Programmed Death-Ligand 1) expression levels to guide immunotherapy decisions such as for predicting response to immune checkpoint inhibitors; typically measured by IHC. AI models could help identify patients who might benefit from checkpoint inhibitors, even when traditional laboratory tests such as IHC provide borderline or discordant results. Patients with high PD-L1 expression benefit from immune checkpoint inhibitors such as pembrolizumab, nivolumab, or atezolizumab, either as monotherapy or in combination with chemotherapy.
Melanoma: ML models may be used for predicting BRAF mutations based on histopathological images, potentially guiding the use of targeted therapies like BRAF and MEK inhibitors.
Breast Cancer: Beyond ER status, ML models may be used for predicting HER2 (Human Epidermal Growth Factor Receptor 2) expression and/or PIK3CA mutations, which may be used for selecting targeted therapies such as trastuzumab for HER2+ cancers or alpelisib for PIK3CA-mutated cases. HER2-positive breast cancer may be treated with HER2-targeted therapies such as trastuzumab, pertuzumab, lapatinib, and trastuzumab deruxtecan. The aforementioned therapies may be combined with chemotherapy for early and advanced disease settings.
In another example, the ML models may be used for predicting an OncotypeDX score, optionally an OncotypeDX recurrence score (ODX), which may be implemented as a multi-gene assay for guiding chemotherapy decisions in early state, ER-positive breast cancer. Patients with low ODX scores typically receive endocrine therapy alone, while those with high scores benefit from the addition of chemotherapy. For premenopausal women, scores in the intermediate range (16-25) may still indicate a benefit from chemotherapy.
Colorectal Cancer: ML models may be used for predicting MSI (Microsatellite Instability) status to determine the likely effectiveness of immunotherapy treatments. MSI-high tumors often respond better to immunotherapy and have distinct prognostic implications. MSI-high colorectal cancers are highly responsive to immune checkpoint inhibitors such as pembrolizumab and nivolumab, which have become preferred treatments in advanced and metastatic cases.
Another example of a marker is TP53 mutation status. TP53 alterations are common in many cancers, shaping tumor biology, progression risk, and therapy responsiveness. While TP53 mutations are prognostic, at the time of filing of the present disclosure, they do not have direct targeted therapies. However, TP53-mutant cancers may be resistant to standard chemotherapy and may benefit from novel experimental agents targeting p53 pathways. TP53 may be considered a prognostic marker.
Lung Cancer: EGFR (Epidermal Growth Factor Receptor) mutation status may be used to predict response to tyrosine kinase inhibitors (TKIs) in non-small cell lung cancer. EGFR-mutant lung cancers may be treated with TKIs such as osimertinib, gefitinib, erlotinib, and/or afatinib. These agents are preferred over chemotherapy in EGFR-mutated non-small cell lung cancer (NSCLC).
Prostate Cancer: NCCN (National Comprehensive Cancer Network) Risk Stratification may be used to categorize patients into low, intermediate, or high risk, influencing treatment choices such as surgery, radiation, or active surveillance. NCCN may serve as the marker described herein. Low-risk patients may be managed with active surveillance, while intermediate- and high-risk cases may be treated with radical prostatectomy, external beam radiation therapy (EBRT), or brachytherapy. Androgen deprivation therapy (ADT) may be added in higher-risk cases.

Other examples of markers, associated cancers, standard laboratory tests, and targeted treatments guided by the marker(s) include:
ALK/ROS1 Rearrangements (Lung Cancer)
   Laboratory test: Often identified by FISH or PCR.
   Clinical Role: Targets for TKIs in non-small cell lung cancer. AI-based morphological signatures may pre-screen patients for confirmatory testing.
   Targeted Treatment: ALK-positive or ROS1-positive lung cancer patients are treated with ALK or ROS1 inhibitors such as crizotinib, ceritinib, alectinib, or lorlatinib.
AR (Androgen Receptor) in Prostate Cancer
   Clinical Role: Guides hormonal therapies; AI could detect functional AR pathway activity from histology, refining treatment decisions beyond standard IHC.
   Targeted Treatment: Androgen deprivation therapy (ADT), including agents such as leuprolide, abiraterone, and enzalutamide, may be used for AR-positive prostate cancer.
BRAF Mutation (Melanoma, Colon Cancer)
   Clinical Role: Predictive biomarker for targeted therapies (e.g., BRAF inhibitors). AI-based classification can flag tumors likely to harbor BRAF mutations, prompting confirmatory molecular testing.
   Targeted Treatment: BRAF-mutant melanoma and colorectal cancer may be treated with BRAF inhibitors (e.g., vemurafenib, dabrafenib) in combination with MEK inhibitors (e.g., trametinib), predicted to improve response rates and prevent resistance.
TMB (Tumor Mutational Burden) and Other Genomic Signatures
   Cancers: Multiple solid tumors.
   Clinical Role: High TMB correlates with improved response to immunotherapy; AI-based morphology can approximate TMB to guide therapy decisions.
   Targeted Treatment: Patients with high TMB often respond well to immune checkpoint inhibitors such as pembrolizumab. TMB is an emerging biomarker in multiple cancers, including lung and bladder cancer.
Gene Expression Signatures (e.g., MammaPrint, Prosigna)
   Laboratory test: RNA-based assays.
   Clinical Role: Provide prognostic and predictive information in breast cancer. AI-driven H&E analysis can approximate or enhance these multi-gene signatures, reducing reliance on costly molecular tests.
   Targeted Treatment: Patients with high-risk MammaPrint scores may receive adjuvant chemotherapy, while low-risk patients may be spared from unnecessary treatment.
Proliferation Markers (e.g., Ki-67)
   Laboratory test: Often measured by IHC in breast, neuroendocrine, and other tumors.
   Clinical Role: Higher Ki-67 levels indicate aggressive tumor behavior; AI-based morphology could gauge proliferative patterns as a surrogate measure.
   Targeted Treatment: In breast cancer, Ki-67 helps guide chemotherapy decisions. In neuroendocrine tumors, high Ki-67 levels indicate the need for aggressive treatment, including platinum-based chemotherapy.

At 204, the image of the histology slide is fed into a machine learning (ML) model trained to predict presence of a selected molecular biomarker. The selected molecular biomarker which the ML model is trained to predict corresponds to the molecular biomarker which the laboratory test is designed to detect.

An exemplary approach for training of the machine learning model is described, for example, with reference to FIG. 3.

Optionally, other data is fed into the ML model, optionally in combination with the image, for example, an indication of a genetic sequence obtained from the tissue, demographic parameters of the subject (e.g., age, gender), and/or medical history of the subject (e.g., history of previous cancers, history of prior treatments, other medical conditions, smoking status). Alternatively, no other data is fed into the ML. The ML may be fed only the histological image, excluding other data.

Optionally, the machine learning model is trained on a training dataset of records. A record includes a sample histology slide of the cancer obtained from a sample subject, and a ground truth indicating positive status or negative status of the molecular biomarker obtained according to the laboratory test. The record may include additional data that is fed into the ML model and/or which is outputted by the ML model, for example, predicted survival optionally according to treatment including the targeted treatment, genetic sequence, and the like.

Exemplary architectures of the ML include, for example, an individual model and/or a pipeline combination of models, for example, neural networks of various architectures (e.g., convolutional, fully connected, deep, encoder-decoder, recurrent, transformer, graph), detector(s), support vector machines (SVM), logistic regression, k-nearest neighbor, decision trees, boosting, random forest, a regressor, and/or any other commercial or open source package allowing regression, classification, dimensional reduction, supervised, unsupervised, semi-supervised, and/or reinforcement learning. Machine learning models may be trained using supervised approaches and/or unsupervised approaches.

The machine learning model(s) described herein may be trained using a variety of advanced techniques to ensure accurate analysis of histopathological images and prediction of biomarker status. While Convolutional Neural Networks (CNNs) are highlighted due to their effectiveness in image recognition and medical image analysis, the model's training and/or architecture is not restricted to this approach. The model may incorporate one or more of the following advanced methodologies to enhance performance, generalization, and interpretability:
Supervised Learning: The model is trained using a labeled dataset containing histopathological images annotated with biomarker statuses, enabling direct learning of patterns associated with molecular classifications.
Semi-Supervised Learning (SSL): Leveraging both labeled and unlabeled data to enhance generalization, particularly useful in medical imaging where fully labeled datasets are often limited. SSL techniques, such as consistency regularization, pseudo-labeling, and contrastive learning, allow the model to learn meaningful representations even with minimal expert annotations.
Transfer Learning: Utilizing pre-trained models from large-scale image datasets (e.g., ImageNet) or foundation models in histopathology, fine-tuning them on domain-specific histopathological data to improve efficiency and performance.
Transformer-Based Architectures: Beyond traditional Vision Transformers (ViTs), transformer models specifically optimized for histopathology, such as hierarchical transformers or hybrid CNN-transformer architectures, can process image patches in a non-local manner, capturing long-range dependencies and contextual relationships crucial for biomarker prediction.
Foundation Models: Leveraging self-supervised learning (SSL) and large-scale pretraining on diverse histopathological datasets, foundation models can provide robust feature representations that generalize well across different biomarker prediction tasks. These models, such as CLIP-like architectures adapted for digital pathology or masked autoencoders, enable zero-shot or few-shot learning for novel biomarkers with minimal additional training.
Multiple Instance Learning (MIL): Particularly well-suited for whole-slide image (WSI) analysis, where individual tiles (instances) within a slide (bag) contribute to the overall biomarker classification. MIL frameworks, including attention-based MIL models, enable the model to learn which regions of the slide are most informative for the prediction task.
Contrastive Learning and Self-Supervised Pretraining: Models trained using contrastive objectives (e.g., SimCLR, MoCo, DINO) can learn robust histopathology-specific representations without requiring extensive labeled datasets. These approaches can be combined with downstream supervised fine-tuning for biomarker prediction.
Ensemble Methods: Combining multiple model predictions (e.g., CNNs, transformers, MIL-based approaches) through techniques like majority voting, weighted averaging, or meta-learning to improve accuracy, robustness, and interpretability.

In at least one embodiment for predicting biomarker status and subsequent patient categorization based on histopathological images, genetic mutations and sequencing data may play a complementary role in validating the predictions made by the ML model. Including genetic sequencing as part of the validation process may provide one or more of the following potential advantages:
Confirm the AI-predicted classifications are correlated with underlying genetic characteristics.
Provide a deeper understanding of the molecular basis behind the divergence between AI-predicted and actual biomarker statuses.
Enhance the AI model's predictive accuracy and utility in clinical settings by incorporating multimodal data sources, including both imaging and genetic information.

Genetic sequencing may be relevant and/or beneficial to the comprehensive application of the disclosure, though the main embodiments do not necessarily depend solely on genetic data.

Given a target cohort of patients with a specific molecular or genetic biomarker indicating treatment eligibility, the following method may be used. Train a model to predict the biomarker status from the patients' diagnostic H&E images. Optionally, use another larger cohort for initial model training, then fine-tune the model on the target cohort using cross-validation to enhance prediction performance. The model's output, the AI score, should accurately predict the biomarker status. As a validation measure, the AUC prediction performance should be above a predefined threshold.

At 206, a score indicating presence of the molecular biomarker associated with the targeted treatment is generated by the machine learning model.

The score may be, for example, a likelihood, optionally a probability, of a positive status or a negative status of the molecular biomarker.

In another example, the score may be for another predefined scale. The predefined scale may correspond to a predefined scale used by a laboratory test to detect the molecular biomarker. For example in records used to train the ML model, the ground truth is within the predefined scale of the laboratory test.

The score, such as likelihood, optionally probability, may be represented as a continuous variable within a range. For example, within the range of 0-1, 0-100, and the like.

Optionally, the ML model generates a predicted survival time. The predicted survival time may be for a scenario in which the subject is treated using the targeted treatment associated with the molecular biomarker for which the ML model generates the probability of the positive status or negative status. Alternatively or additionally, the ML model may generate a predicted survival time for a scenario in which the subject is treated with treatment that is different than the targeted treatment, i.e., excludes the targeted treatment. Alternatively or additionally, the ML model may generate a predicted survival time for a scenario in which the marker is implemented as a prognostic marker.

The training dataset of the machine learning model may further include a ground truth indicating the predicted survival time(s).

At 208, an indication of the positive status or the negative status of the molecular biomarker obtained by a laboratory test for presence of the molecular biomarker on the cancer, is provided, for example, accessed, received, and/or obtained. The molecular biomarker which the laboratory test is designed to detect corresponds to the molecular biomarker which is predicted by the ML model described herein.

In embodiments in which the marker is implemented as a prognostic marker, a state of the prognostic marker may be obtained based on the laboratory test, for example, a prediction of the survival time.

The molecular biomarker may be, for example, a validated gene signature, gene expression status, mutation status, and the like⁹.

Optionally, a score is obtained for the molecular biomarker. The score may positive status or negative status of the molecular biomarker, for example, a value of the score above a threshold indicates positive status, and a value of the score below the threshold indicates negative status. For example, for Oncotype DX, the threshold may be 26. In another example, the score may be defined according to the predefined scale. The predefined scale may correspond to the predefined scale used by the trained ML model.

The laboratory test may be based on visual depiction of the molecular biomarker associated with the targeted treatment.

In an example, the laboratory test is based on immunohistochemistry. For example, the molecular biomarker may be an antibody marker linked to a fluorescent dye that is visible using immunofluorescence. In another example, the laboratory test is based on RNA sequence.

The molecular biomarker may be a receptor associated with the targeted treatment. Alternatively or additionally, the biomarker may be another structure other than a receptor, for example, PDL1 which is a protein but not a receptor.

The positive status or negative of the molecular biomarker may be according to clinical guidelines of the specific molecular biomarker. For example, when the molecular biomarker is HER2, multiple parameters may be checked to determine the HER2 category (0,1,2,3), including IHC staining intensity, extent, and texture. HER2 status is defined as being negative for categories 0 and 1, positive for category 3. For category 2, a FISH test is then done. FISH above or below a threshold determines the status. It is noted that clinical guidelines and/or cutoffs may be dynamic, changing over the years.

It is noted that the positive or negative status of the molecular biomarker may be obtained from a different physical sample of cancer than the sample of cancer tissue used in the histology slide. However, it may be assumed that the same status of the molecular biomarker is the same for the different physical samples of tissue since they are from the same cancer tissue from the same subject. For example, a breast cancer patient is diagnosed using a lumpectomy sample. This sample is split into blocks. Each block is split into slides. For one of these blocks, 2 slides are stained for H&E, and another one for ER. For another block, 1 slide is stained for H&E and one for ER. In this example, there are 3 slides from a single patient, where each slide from a different block. Each block has its own independent ER status.

Examples of laboratory tests for biomarkers of cancer include:
1. Immunohistochemistry (IHC)
   Example Biomarkers:
      HER2 (Breast & Gastric Cancer)→Determines eligibility for trastuzumab (Herceptin)
      PD-L1 (Lung & Other Cancers)→Predicts response to immune checkpoint inhibitors (e.g., pembrolizumab)
      ER/PR (Breast Cancer)→Determines eligibility for hormone therapy (e.g., tamoxifen, aromatase inhibitors)
2. Fluorescence In Situ Hybridization (FISH)
   Example Biomarkers:
      HER2 amplification (Breast Cancer)→Confirms HER2-targeted therapy eligibility
      ALK rearrangement (Non-Small Cell Lung Cancer)→Determines response to ALK inhibitors (e.g., crizotinib)
3. Next-Generation Sequencing (NGS)
   Example Biomarkers:
      EGFR mutations (Lung Cancer)→Indicates response to EGFR inhibitors (e.g., osimertinib)
      BRAF V600E mutation (Melanoma, Colorectal Cancer)→Suggests use of BRAF inhibitors (e.g., vemurafenib)
      BRCA1/2 mutations (Breast, Ovarian Cancer)→Guides use of PARP inhibitors (e.g., olaparib)
4. Polymerase Chain Reaction (PCR) & RT-PCR
   Example Biomarkers:
      KRAS mutations (Colorectal & Lung Cancer)→Determines if anti-EGFR therapy (e.g., cetuximab) is effective
      IDH1/IDH2 mutations (Gliomas, Leukemia)→Helps in selecting IDH inhibitors (e.g., ivosidenib)
5. Liquid Biopsy (Circulating Tumor DNA-ctDNA Tests)
   Example Biomarkers:
      EGFR T790M mutation (Lung Cancer)→Identifies resistance to first-line EGFR inhibitors and guides use of osimertinib
      PIK3CA mutations (Breast Cancer)→Helps select patients for PI3K inhibitors (e.g., alpelisib)
6. Flow Cytometry
   Example Biomarkers:
      CD19/CD20/CD22 (Lymphomas & Leukemias)→Helps guide monoclonal antibody therapy (e.g., rituximab for CD20+B-cell lymphoma)
7. Microsatellite Instability (MSI) & Mismatch Repair Deficiency (dMMR) Testing
   Example Biomarkers:
      MSI-High/dMMR (Colorectal, Endometrial Cancer)→Predicts response to immune checkpoint inhibitors (e.g., pembrolizumab)

At 209, a threshold (denoted T) may be computed. The threshold may be used for classifying the cancer according to the score generated by the ML model. The threshold may be used for determining whether the score generated by the ML model is discordant with respect to the outcome determined according to the laboratory test, for classifying the cancer into the first or second categories (or the third or fourth categories), as described herein. For examples, scores generated by the ML model above T are classified as “positive” and scores below T as “negative.” The “positive” or “negative” outcomes determined by applying the threshold to the scores generated by the ML model may be compared to the “positive” or “negative” outcomes obtained based on the laboratory test, to determine whether the cancer is a discordant case, and classified into the first or second category.

The threshold may be computed by maximizing the balanced accuracy of the prediction. It is noted that other methods may be used to compute the threshold depending on the application.
Accuracy/ROC-Based Selection
   Choose T that maximizes an evaluation metric such as accuracy, Youden's J statistic (sensitivity+specificity−1), or AUC-ROC performance, or F1.
   Example: If AI predicts ER status, test various values of T on a validation set and pick the one that yields the highest classification accuracy against IHC-labeled ground truth.
Prevalence Matching
   Select T such that the AI-positive rate aligns with the known biomarker prevalence in the population.
   Example: If 70 percent of patients are typically ER-positive by IHC, adjust T so that AI classifies approximately 70 percent of cases as ER-positive.
Cost-Benefit Considerations
   Optimize T to balance the risks of false positives (unnecessary treatment) and false negatives (missed therapy opportunity).
   Example: In HER2-targeted therapy, setting T higher reduces overtreatment but risks missing borderline HER2-positive cases.
Calibration
   First adjust the model's output to represent true probability estimates using calibration techniques. Examples: Platt scaling or isotonic regression.
   Example: If an AI model predicts HER2 status, post-training calibration ensures that an AI score of 0.8 represents an 80 percent probability of HER2 positivity.
In many cases, a single threshold does not provide enough granularity. Instead, two cutoffs T1 and T2 (T1<T2) can be used to define three groups:
   Scores below T1→“Low” (negative)
   Scores between T1 and T2→“Intermediate” (uncertain/requires additional testing)
   Scores above T2→“High” (positive)

At 210, in response to the likelihood, optionally the probability, being equal to or greater than the threshold and the molecular biomarker according to the laboratory test having a negative status, the cancer is classified as a first category.

The first category may indicate that the cancer is likely to respond to the targeted treatment despite the negative status of the molecular biomarker by the laboratory test.

In embodiments in which the cancer is classified into the first category, the marker includes a prognostic marker, and/or the predicted survival time is generated by the machine learning model and/or the laboratory test generates a state of the prognostic marker, the predicted survival time generated by the machine learning model may be provided in place of the prediction of the survival time based on the laboratory test. The first category may indicate that the prediction of the machine learning model is more accurate than of the laboratory test.

Subjects classified into the first category may be considered as having a distinctive tumor biology than subjects with the same biomarker score obtained from the laboratory test. The tumor biology of subjects classified into the first category may be more similar to the tumor biology corresponding to the score predicted by the ML model than to the score of the actual marker value obtained by the laboratory test.

The threshold may be selected for indicating a positive presence of the molecular biomarker by the ML model even though the laboratory test indicated the negative status. The threshold of probability may be set at, for example, about 50%, or 60%, or 75%, or 80%, or 90%, or 95%, or 100%, or other values, or within the range of about 50-100%, or about 60%-90%, or about 70-80%, or other ranges.

At 212, in response to the classification of the cancer as the first category, the subject may be treated using the targeted treatment.

It is noted that treating the subject using the targeted treatment is different than existing clinical guidelines, which consider the laboratory test as the gold standard. Such existing approaches would ignore the results generated by the ML model and/or place not value on the results generated by the ML model, assuming that the results generated by the ML model are erroneous since they contradict the gold standard laboratory test results. In contrast, as described herein, Inventors discovered that the results generated by the ML model may indicate whether the subject should be treated with the targeted treatment.

Alternatively or additionally, at 214, in response to the classification of the cancer into the first category, the subject may be excluded from a clinical trial with inclusion criteria indicating negative status of the molecular biomarker according to the laboratory test.

Excluding the subject from the clinical trial may reduce inaccuracies that may arise due to the status of the subject, where the subject is to be treated with the targeted treatment based on the score generated by the ML model even though the laboratory test indicates negative status of the molecular biomarker. The subject does not necessarily meet the full inclusion criteria, since the status of the molecular biomarker is shown to be “positive” (i.e., score greater than the threshold) according to the outcome generated by the ML model.

At 216, in response to the likelihood, optionally the probability, being less than the threshold and the molecular biomarker according to the laboratory test having a positive status, the cancer is classified as a second category.

The same threshold used to classify the cancer into the first category may be used for classification into the second category. The threshold may be used as a binary classification, where the cancer is classified into the first category when the probability is above the threshold, and classified into the second category when the probability is below the threshold.

The second category may indicate that the cancer is unlikely to respond to the targeted treatment despite the positive status of the molecular biomarker.

In embodiments in which the cancer is classified into the second category, the marker includes a prognostic marker, and/or the predicted survival time is generated by the machine learning model and/or the laboratory test generates a state of the prognostic marker, the predicted survival time generated by the machine learning model may be provided in place of the prediction of the survival time based on the laboratory test. The second category may indicate that the prediction of the machine learning model is more accurate than of the laboratory test.

Subjects classified into the second category may be considered as having a distinctive tumor biology than subjects with the same biomarker score obtained from the laboratory test. The tumor biology of subjects classified into the second category may be more similar to the tumor biology corresponding to the score predicted by the ML model than to the score of the actual marker value obtained by the laboratory test.

At 218, in response to the classification of the cancer into the second category, the subject may be treated with another treatment predicted to be effective for the cancer. The other treatment is different than the target treatment, i.e., the other treatment excludes the targeted treatment. The other treatment may be, for example, traditional chemotherapy, radiation therapy, and/or surgical therapy.

It is noted that treating the subject using the other treatment rather than the targeted treatment is different than existing clinical guidelines, which consider the laboratory test as the gold standard. Such existing approaches would ignore the results generated by the ML model and/or place not value on the results generated by the ML model, assuming that the results generated by the ML model are erroneous since they contradict the gold standard laboratory test results. In contrast, as described herein, Inventors discovered that the results generated by the ML model may indicate whether the subject should be treated with the other treatment as opposed to the targeted treatment.

Alternatively or additionally, at 220, in response to the classifying the cancer into the second category, the subject may be excluded from a clinical trial with inclusion criteria indicating positive status of the molecular biomarker.

Excluding the subject from the clinical trial may reduce inaccuracies that may arise due to the status of the subject, where the subject is to be treated with another treatment (different from the targeted treatment) based on the score generated by the ML model even though the laboratory test indicates positive status of the molecular biomarker. The subject does not necessarily meet the full inclusion criteria, since the status of the molecular biomarker is shown to be “negative” (i.e., score lower than the threshold) according to the outcome generated by the ML model even though the laboratory test indicates “positive”.

At 222, in response to the likelihood, optionally the probability, being equal to or greater than the threshold and the molecular biomarker according to the laboratory test indicating a positive status, the cancer is classified into a third category.

The same threshold used for the first and second categories may be used for the third category. Alternatively, a different threshold may be selected.

The third category indicates that both the outcome generated by the ML model and the laboratory test are aligned, indicating positive status of the molecular biomarker.

The alignment of both the ML model and the laboratory test may indicate a better predicted outcome for the subject when treated using the targeted therapy.

A higher value of the probability may indicate a better predicted outcome for the subject in response to being treated using the targeted therapy.

At 224, the subject may be treated, excluded/included in the clinical trial, and/or provided with a prognosis, according to the results of the laboratory test of the marker. For example, the subject may be treated using the targeted therapy.

At 226, in response to the likelihood, optionally the probability, being less than the threshold and the molecular biomarker according to the laboratory test indicating a negative status, the cancer is classified into a fourth category.

The same threshold used for the first and/or second and/or third categories may be used for the fourth category. Alternatively, a different threshold may be selected.

A lower values of the probability may indicate a worse predicted outcome for the subject in response to being treated with the targeted therapy.

At 228, the subject may be treated, excluded/included in the clinical trial, and/or provided with a prognosis, according to the results of the laboratory test of the marker. For example, the subject may be treated using another therapy different than the target therapy, i.e., the subject is not treated with the targeted therapy according to the results of the laboratory test.

At 230, other examples of use cases and/or additional details of aforementioned use cases of classification of cancer using at least one embodiment described herein, related to discordance between the AI model and the laboratory test, are now described:

One set of use cases relates to Clinical Decision-Making & Patient Management:
Refining Treatment Selection
   Patients classified as discordant may require alternative treatment strategies, as their tumor biology may not align with standard biomarker-driven therapy decisions.
   Example: AI predicts a patient's tumor as ER-negative, but IHC classifies it as ER-positive. If gene expression confirms a lack of ER pathway activity, the patient may be better suited for chemotherapy rather than endocrine therapy.
Personalized Therapy Adjustment
   Patients with discordant profiles may not respond as expected to standard therapies. Identifying these cases early could prevent unnecessary or ineffective treatments.
   Example: HER2-positive patients by IHC but predicted negative by AI may have low HER2 pathway activation, indicating they might not benefit from anti-HER2 therapies.
Flagging Patients for Additional Testing
   Discordant cases may warrant additional molecular assays (e.g., RNA sequencing, genomic profiling) to clarify true tumor biology.
   Example: An ER-positive but AI-predicted ER-negative case could be re-evaluated using a gene expression panel to determine if ER signaling is functionally active.
Minimizing Misdiagnoses in Pathology
   AI-based predictions can serve as a secondary quality check to flag potential errors in standard biomarker assessment.
   Example: If an AI model flags a HER2-negative tumor as HER2-positive, the pathologist may choose to reassess the IHC or FISH test results.

Another set of use cases relates to Enhancing Clinical Trial Design & Drug Development:
Stratification of Patients for Clinical Trials
   Clinical trials often rely on biomarker-based inclusion criteria. Discordant cases may represent a biologically distinct subgroup that should be included or excluded from specific trials.
   Example: In a trial testing endocrine therapy, ER-positive patients with AI-predicted ER negativity could be analyzed separately to determine if they have differential responses.
Exclusion of High-Risk Mislabeled Patients in Trials
   Patients with high AI-marker discrepancies may introduce confounding factors in clinical trials and should be excluded from efficacy analyses.
   Example: If a clinical trial is testing a drug on HER2-positive patients, but AI predicts some as functionally HER2-negative, their exclusion could improve trial outcome accuracy.

Yet another set of use cases relates to Advancing Cancer Biology Research:
Identifying New Tumor Subtypes
   Discordant cases may reveal previously unrecognized tumor subtypes with distinct molecular or morphological characteristics.
   Example: Some ER-positive but AI-predicted ER-negative tumors may share characteristics with basal-like breast cancers, even though standard tests classify them differently.
Understanding Tumor Plasticity & Evolution
   Tumors undergo molecular evolution over time, which may alter biomarker expression. Discordant cases could capture dynamic changes missed by standard tests.
   Example: An AI-predicted biomarker shift could indicate early resistance mechanisms, such as HER2 downregulation in metastatic settings.
Investigating Pathway Activation vs. Protein Expression
   Some biomarkers indicate protein expression but do not reflect actual functional pathway activation. AI-based discordance analysis can highlight cases where expression does not correlate with function.
   Example: HER2-positive tumors by IHC but functionally HER2-negative by AI could indicate HER2 pathway inactivity due to co-occurring genetic alterations.

Reference is now made to FIG. 3, at 302, a sample histology slide of a cancer obtained from a sample subject is obtained. Exemplary histology slides are described, for example, with reference to 202 of FIG. 2.

At 304, a score indicative of presence of a selected molecular biomarker is obtained according to a laboratory test. The score may indicate positive status or negative status of the selected molecular biomarker. Additional exemplary details of obtaining an indication of the molecular biomarker from a laboratory test are described, for example, with reference to 206 of FIG. 2.

At 306, other data may be obtained, for example, survival time of the subject after a time when the sample of cancer was obtained from the subject, demographic data, genetic data, and the like. Alternatively, other data is not obtained. Exemplary details of other data are described, for example, with reference to 204 of FIG. 2.

At 308, a record is created. The record includes the sample histology slide, and optionally additional data such as demographic data and/or genetic data.

The record may further include a ground truth indicating the status of the molecular biomarker obtained according to the laboratory test, for example, the score and/or the positive status or negative status. Optionally, the ground truth includes the survival time.

At 310, features described with reference to 302-308 are iterated for different sample histology slides of different subjects. The iterations are performed for creating multiple records, which are included in a training dataset.

Optionally, each training dataset includes histology images of a same type of cancer with indication of a same type of molecular biomarker, from different subjects. This enables training one more machine learning models that are each specific for predicting presence of a specific molecular biomarker in a specific cancer—examples are provided herein.

At 312, the machine learning model is trained on the training dataset.

Instead of training the model on the entire dataset and testing on the same data (which can lead to over-optimistic results), a cross-validation may be implemented. Cross-validation is a method to help ensure that the model performs well on unseen data by repeatedly training and testing on different parts of the dataset. The following cross-validation may be used:
Split the Data: First, the dataset is divided into several equal parts (or “folds”). For example, for a 5-fold cross-validation approach, the data is split into 5 equal parts.
Training and Testing Multiple Times: The model is then trained and tested a number of times according to the number of equal parts, for example, 5 times. Each time, one part is held out as a test set while the model is trained on the remaining parts, for example, 4 parts. This means every part of the data gets used for testing once.
Evaluate and Average: After each round, the model's performance is measured (e.g., accuracy). By the end, these measurements may be averaged to get an overall performance score. This average may be generally more reliable than testing on a single split because it shows how well the model performs across different subsets of data.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments in a not necessarily limiting fashion.

Inventors developed, tested, and validated deep-learning models using The Cancer Genome Atlas Breast Cancer (TCGA-BRCA) cohort. The Cancer Genome Atlas Breast Cancer (TCGA-BRCA) dataset, publicly available and spanning 40 sites, comprises 3,112 H&E slides from 1,097 patients diagnosed between 1988 and 2013. It includes both formalin-fixed paraffin-embedded and frozen tissue sections. Inventors utilized both diagnostic and frozen sections to enrich the training data, aiming to enhance diversity and model generalizability. The inference was done on the diagnostic slides. Slides with insufficient tissue were excluded from the analysis. In total, 2,576 diagnostic slides from 1,040 TCGA patients were used in this study for training and analysis.

Inventors randomly split the TCGA patients into TCGA-train (75% of patients) and TCGA-test (25% of patients) sets. The TCGA-train was divided into five equal folds at the patient level for training and validating the models in a cross-validation approach, focusing on predicting the ER status from the H&E images. The models output a prediction score per patient, denoted herein as the likelihood, but also referred to herein as an artificial intelligence (AI) score, ranging from 0 to 1, which predicts the likelihood of ER positivity. The models achieved a cross-validation AUC per slide of 0.910 for ER, indicating a strong predictive capability from H&E whole slide images. After cross-validation, Inventors locked the models and evaluated them on the TCGA-test data, achieving AUCs of 0.944.

The high performance indicates that, in most cases, ER-negative patients had low AI scores, and ER-positive patients had high AI scores. However, the performance was not perfect, because a small portion of the ER-negative patients had high AI scores, while a small portion of ER-positive patients had low AI scores.

Referring now back to FIG. 4, graphs 402 comparing the gene expression of four groups: ER-negative patients, ER-positive patients, ER-negative AI-high, and ER-positive AI-low, are presented.

FIG. 4 presents the mean expression of representative genes in ER-negative and ER-positive patients, and in subgroups with divergent AI scores: ER-negative patients with high AI scores (ER− AI+), and ER-positive patients with low AI scores (ER+ AI−). The ER-negative AI-high subgroup was selected based on the top 5% of AI scores across ER-negative patients, while the ER-positive AI-low subgroup was determined by the bottom 5% of AI scores among ER-positive patients. The bars include 95% confidence intervals for each group. It is noted that other percentages may be selected.

The results of the experiment, including results presented in FIG. 4, indicate that patients with ER-negative and high AI scores exhibited a significantly different genetic signature compared to the rest of the ER-negative patients. Similarly, ER-positive AI-low patients showed a significantly different genetic signature than the rest of the ER-positive patients. This may indicate that although the models were trained to predict the ER status, the learned morphological signal may be associated with a significantly different genetic expression, suggesting that the models uncovered a distinctive tumor subtype.

Inventors conducted another experiment to evaluate the survival time of patients with low and high AI scores, comparing them to the ER status and other clinical variables.

Referring now back to FIGS. 5A-D, graphs comparing patient prognosis for ER status and predicted morphological signal, obtained during an experiment, are presented.

FIG. 5A presents a Kaplan-Meier analysis of TCGA patient survival time, stratified by ER+ AI+, ER+ AI−, and ER−. The analysis indicates that ER-positive patients with low AI scores may exhibit a prognostic behavior similar to ER-negative patients. This may suggest that although these patients have ER-positive expression by IHC, their tumors are not influenced by these receptors. FIG. 5B presents a proportional hazard ratio measurement for each group of ER-positive patients across different AI score ranges, compared to the survival time of all ER-positive patients. This may indicate a tendency towards lower prognosis with lower AI scores. FIG. 5C and FIG. 5D include a Multivariate Cox Proportional Hazard analysis of various clinical variables, with and without the AI score, in relation to patient survival time.

The results, including the results presented in FIGS. 5A-D, indicate that ER-positive patients with low AI scores may have a significantly worse prognosis than the rest of the ER-positive group. Interestingly, these patients may exhibit a prognosis similar to that of ER-negative patients. This may suggests that ER-positive AI-low tumors, despite having ER receptors present in IHC staining, may not be influenced by these receptors. Inventors hypothesize that ER-positive AI-low tumors are effectively functioning as ER-negative tumors. In other words, the AI scores may predict the ER dependency rather than the ER status, although trained to predict the ER status. The multivariate analysis presented in FIG. 5D further demonstrates that the AI score may add prognostic value beyond clinical variables and ER status.

Inventors conducted yet another experiment.

Inventors created a dataset of H&E-stained tissue microarray (TMA) images from 5,300 breast cancer patients to investigate whether tumor morphology alone could reveal molecular profiles. This work was among the first to demonstrate that deep learning models can be applied to tumor morphology seen in H&E images to predict molecular marker expression, including ER, PR, Her2, and 18 other markers3. Inventors have expanded the data collection significantly, gathering over 35,000 H&E whole slide images from various sources. This expanded dataset has supported a broader range of studies, including models for predicting PD-L1 8, ER, PR, Her2¹⁸, and OncotypeDX19 scores in breast cancer. Additionally, Inventors developed and implemented a quality assurance (QA) alert system based on H&E analysis at Carmel Medical Center. This system flagged 31 receptor status misdiagnoses (about 4-6% of cases reviewed), some of which impacted treatment eligibility 1⁸.

An AUC of 92-95% was obtained for the ML models trained for predicting ER status. While most cases were concordant between AI predictions and IHC results, some cases were discordant.

Referring now back to FIG. 6, which includes graph 602 depicting the discordant cases. These discordant cases included tumors identified as ER-negative by IHC with high AI scores, and tumors labeled ER-positive by IHC but receiving low AI scores according to at least one embodiment described herein. The AI model, trained to predict ER status from H&E images, generated AI scores for patients in a held-out test set (TCGA-BRCA). The scores are displayed in two box plots—a first box plot 604 for ER-negative patients and a second box plot 606 for ER-positive patients—highlighting the presence of ER-positive patients with low AI scores and ER-negative patients with high AI scores.

Inventors discovered that these discrepancies reveal biological differences overlooked by conventional IHC assessments.

To explore the biological basis of discordances between AI predictions and IHC results, Inventors examined gene expression data from the TCGA breast cancer cohort. The goal was to determine if AI-derived scores capture biologically relevant information beyond traditional IHC assessments. Inventors developed a gene expression-based score that quantifies how closely each tumor's transcriptome aligns with typical ER-positive or ER-negative profiles, ranging from 0 (strongly ER-negative-like) to 1 (strongly ER-positive-like)²⁰.

Inventors stratified samples by their AI-derived ER scores into quantiles (0-20%, 20-40%, 40-60%, 60-80%, and 80-100%).

Referring now back to FIGS. 7A-7F, graphs comparing gene expression-based scores within each quantile between IHC-determined ER-positive and ER-negative cases as part of an experiment conducted by Inventors, are presented.

FIG. 7A is a graph based on the model trained to predict breast cancer ER status. An analysis of the graph revealed a consistent trend: as the AI-derived ER score increased, the gene expression-based score also rose, regardless of IHC-determined ER status. Strikingly, ER-negative cases with high AI scores exhibited higher gene expression-based scores than ER-positive cases with low AI scores. This suggests that these tumors exhibit a transcriptional state aligning with the AI scores rather than the IHC classification, potentially indicating that the AI models predict ER pathway activity. FIG. 7B is a graph based on the model trained to predict HER2 in breast cancer status. FIG. 7C is a graph based on the model trained to predict status of EGFR in lung cancer. FIG. 7D is a graph based on the model trained to predict status of a NCCN risk group in prostate cancer. FIG. 7E is a graph based on the model trained to predict status of BRAF in colon cancer. FIG. 7F is a graph based on the model trained to predict status of TP53 in colon cancer. For FIGS. 7A-7F, a horizontal axis depicts the AI score quantiles for each model. A vertical axis displays the gene expression-derived score, ranging from 0 to 1, which quantifies each tumor's similarity to gene expression profiles of negative and positive groups corresponding to each marker.

Across these molecular biomarkers, AI-derived scores consistently correlated with gene expression patterns, often independent of IHC or biomarker status. These findings provide evidence that the AI model described herein, trained exclusively on H&E images, captures morphological features reflective of underlying gene expression profiles.

To investigate the clinical relevance of these findings, Inventors analyzed how AI-derived scores relate to patient outcomes and treatment responses, specifically focusing on prognosis in ER-positive and ER-negative breast cancer patients, stratified using the AI model trained to predict the ER status.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant machine learning models will be developed and the scope of the term machine learning model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Pantanowitz, L. et al. An artificial intelligence algorithm for prostate cancer diagnosis in whole slide images of core needle biopsies: a blinded clinical validation and deployment study. Lancet Digit Health 2, e407-e416 (2020).
2. Campanella, G. et al. Clinical-grade computational pathology using weakly supervised deep learning on whole slide images. Nat. Med. 25, 1301-1309 (2019).
3. Shamai, G. et al. Artificial Intelligence Algorithms to Assess Hormonal Status From Tissue Microarrays in Patients With Breast Cancer. JAMA Network Open vol. 2 e197700 Preprint at https://doi(dot)org/10(dot)1001/jamanetworkopen(dot)2019(dot)7700(2019).
4. Naik, N. et al. Deep learning-enabled breast cancer hormonal receptor status determination from base-level H&E stains. Nat. Commun. 11, 5727 (2020).
5. Rawat, R. R. et al. Deep learned tissue ‘fingerprints’ classify breast cancers by ER/PR/Her2 status from H&E images. Sci. Rep. 10, 7275 (2020).
6. Gamble, P. et al. Determining breast cancer biomarker status and associated morphological features using deep learning. Communications Medicine 1, 1-12 (2021).
7. Bychkov, D. et al. Deep learning identifies morphological features in breast cancer predictive of cancer ERBB2 status and trastuzumab treatment efficacy. Sci. Rep. 11, 4037 (2021).
8. Shamai, G. et al. Deep learning-based image analysis predicts PD-L1 status from H&E-stained histopathology images in breast cancer. Nat. Commun. 13, 6753 (2022).
9. Tamoxifen for early breast cancer: an overview of the randomised trials. Early Breast Cancer Trialists' Collaborative Group. Lancet 351, 1451-1467 (1998).
10. Sparano, J. A. Adjuvant chemotherapy guided by a 21-gene expression assay in breast cancer. N. Engl. J. Med. 379, 111-121 (2018).
11. Hanahan, D. & Weinberg, R. A. The Hallmarks of Cancer Review. Cell 100, 57-70 (2000).
12. Herbst, R. S. et al. Atezolizumab for first-line treatment of PD-L1-selected patients with NSCLC. N. Engl. J. Med. 383, 1328-1339 (2020).
13. Inda, M. A. et al. Estrogen receptor pathway activity score to predict clinical response or resistance to neoadjuvant endocrine therapy in primary breast cancer. Mol. Cancer Ther. 19, 680-689 (2020).
14. Chen, M. et al. Classification and mutation prediction based on histopathology H&E images in liver cancer using deep learning. NPJ Precis. Oncol. 4, 14 (2020).
15. Amgad, M. et al. A population-level digital histologic biomarker for enhanced prognosis of invasive breast cancer. Nat. Med. 30, 85-97 (2024).
16. Howard, F. M. et al. Integration of clinical features and deep learning on pathology for the prediction of breast cancer recurrence assays and risk of recurrence. NPJ Breast Cancer 9, 25 (2023).
17. Spratt, D. E. et al. Artificial intelligence predictive model for hormone therapy use in prostate cancer. NEJM Evid. 2, EVIDoa 2300023 (2023).
18. Clinical Validation and Utility of ER, PR, and Her2/ERBB2 Status Prediction in Breast Cancer Using Deep Learning on H&E-Stained Slides.
19. Shamai, G. et al. Abstract 5354: Prediction of OncotypeDX high risk group for chemotherapy benefit in breast cancer by deep learning analysis of hematoxylin and eosin-stained whole slide images. Cancer Res. 83, 5354-5354 (2023).
20. Gong, T. & Szustakowski, J. D. DeconRNASeq: a statistical framework for deconvolution of heterogeneous tissue samples based on mRNA-Seq data. Bioinformatics 29, 1083-1085 (2013).

What is claimed is:

1. A computer implemented method of classifying a cancer for treatment and/or management thereof, comprising:
feeding an image of a histology slide of a cancer obtained from a subject into a machine learning (ML) model;
obtaining a score indicative of a probability of a positive status or a negative status of a marker associated with a targeted treatment from the ML model;
accessing an indication of the positive status or the negative status of the marker obtained by a laboratory test for presence of the marker on the cancer;
computing a threshold for determining whether the score generated by the ML model is discordant with respect to the indication determined according to the laboratory test;
in response to the score being equal to or greater than a threshold and the negative status of the marker according to the laboratory test, classifying the cancer as a first category; and
in response to the score being less than the threshold and the positive status of the marker according to the laboratory test, classifying the cancer as a second category.

2. The computer implemented method of claim 1, wherein the marker is selected from: a molecular biomarker, a genomic marker, and a prognostic marker.

3. The computer implemented method of claim 1, wherein the cancer comprises breast cancer, the marker comprises an estrogen receptor (ER), and targeted treatment is designed for blocking and/or interfering with function of the ER, wherein the targeted treatment is selected from: selective estrogen receptor modulator, aromatase inhibitor, ovarian suppression therapy, AKT inhibitors, CDK 4/6 inhibitors, mTor inhibitors, PI3K inhibitors, and antibody-drug conjugates (ADCs).

4. The computer implemented method of claim 1, wherein the cancer, marker, and target treatment are selected from the following sets:
{breast, HER2, HER2 targeted therapy selected from trastuzumab, pertuzumab, lapatinib, and trastuzumab deruxtecan},
{breast, the marker is determined via the laboratory test of oncotypeDx recurrence score, low ODX scores are treated with endocrine therapy alone and high ODX scores are treated with the addition of chemotherapy},
{prostate, national comprehensive cancer network (NCCN) risk classification, low-risk is managed with active surveillance, intermediate- and high-risk are treated with radical prostatectomy and/or external beam radiation therapy (EBRT) and/or or brachytherapy and/or androgen deprivation therapy (ADT),
{lung, epidermal growth factor receptor (EGFR), tyrosine kinase inhibitors (TKIs)},
{colon, microsatellite instability (MSI), immune checkpoint inhibitors},
{lung or melanoma or head and neck or bladder, programmed death-ligand 1 (PD-L1), immune checkpoint inhibitors},
{non-small cell lung cancer, ALK/ROS1 rearrangements, ALK or ROS1 inhibitors},
{prostate cancer, androgen receptor (AR), androgen deprivation therapy (ADT)},
{melanoma or color, BRAF mutation, BRAF inhibitors and/or MEK inhibitors},
{solid tumor, tumor mutational burden (TMB), immune checkpoint inhibitors},
{breast cancer, mammaprint score, adjuvant chemotherapy},
{neuroendocrine tumors, Ki-67, platinum-based chemotherapy}.

5. The computer implemented method of claim 1, wherein the first category indicates that the cancer is likely to respond to the targeted treatment despite negative status of the marker, and the second category indicates that the cancer is unlikely to respond to the targeted treatment despite positive status of the marker.

6. The computer implemented method of claim 1, further comprising in response to the classifying the cancer as the first category, treating the subject using the targeted treatment.

7. The computer implemented method of claim 1, further comprising in response to the classifying the cancer as the second category, treating the subject with a second treatment predicted to be effective for the cancer, wherein the second treatment excludes the targeted treatment.

8. The computer implemented method of claim 1, further comprising in response to the classifying the cancer as the first category, excluding the subject from a clinical trial with inclusion criteria indicating negative status of the molecular biomarker.

9. The computer implemented method of claim 1, further comprising in response to the classifying the cancer as the second category, excluding the subject from a clinical trial with inclusion criteria indicating positive status of the molecular biomarker.

10. The computer implemented method of claim 1, wherein the image excludes visual depiction of the marker associated with the targeted treatment.

11. The computer implemented method of claim 1, wherein the laboratory test is based on visual depiction of the marker associated with the targeted treatment.

12. The computer implemented method of claim 1, wherein the histology slide includes a slice of the cancer stained with a hematoxylin and eosin (H&E) stain.

13. The computer implemented method of claim 1, wherein the laboratory test includes immunohistochemistry.

14. The computer implemented method of claim 1, further comprising:
creating a training dataset of a plurality of records, wherein a record includes a sample histology slide of the cancer obtained from a sample subject, and a ground truth indicating positive status or negative status of the marker obtained according to the laboratory test; and
training the machine learning model on the training dataset.

15. The computer implemented method of claim 1, wherein:
the marker comprises a prognostic marker,
obtaining the score of the positive status or negative status comprises obtaining a predicted survival time as an outcome of the machine learning model,
wherein accessing comprises accessing a prediction of the survival time based on the laboratory test, and
in response to the cancer being classified as the first category or second category, providing the predicted survival time generated by the machine learning model in place of the prediction of the survival time based on the laboratory test.

16. The computer implemented method of claim 15, further comprising:
creating a training dataset of a plurality of records, wherein a record includes a sample histology slide of the cancer obtained from a sample subject, and a first ground truth indicating positive status or negative status of the marker obtained according to the laboratory test, and a second ground truth indicating survival time; and
training the machine learning model on the training dataset.

17. The computer implemented method of claim 1, wherein the machine learning model is only fed the image of the histology slide of the cancer, excluding other data.

18. A system for classifying a cancer for treatment and/or management thereof, comprising:
at least one processor executing a code for:
feeding an image of a histology slide of a cancer obtained from a subject into a ML model;
obtaining a score indicative of a probability of positive status or negative status of a marker associated with a targeted treatment from the ML model;
accessing an indication of the positive status or the negative status of the marker obtained by a laboratory test for presence of the marker on the cancer;
computing a threshold for determining whether the score generated by the ML model is discordant with respect to the indication determined according to the laboratory test;
in response to the score being equal to or greater than a threshold and the negative status of the marker according to the laboratory test, classifying the cancer as a first category; and
in response to the score being less than the threshold and positive status of the marker according to the laboratory test, classifying the cancer as a second category.

19. A non-transitory medium storing program instructions for classifying a cancer for treatment and/or management thereof, which when executed by at least one processor, cause the at least one processor to:
feed an image of a histology slide of a cancer obtained from a subject into a ML model;
obtain a score indicative of a probability of positive status or negative status of a marker associated with a targeted treatment from the ML model;
access an indication of the positive status or the negative status of the marker obtained by a laboratory test for presence of the marker on the cancer;
compute a threshold for determining whether the score generated by the ML model is discordant with respect to the indication determined according to the laboratory test;
in response to the score being equal to or greater than a threshold and the negative status of the marker according to the laboratory test, classify the cancer as a first category; and
in response to the score being less than the threshold and the positive status of the marker according to the laboratory test, classify the cancer as a second category.
